(19) United States
(12) Patent Application Publication
(10) Pub. No.: US 2010/0041885 A1
(43) Pub. Date: Feb. 18, 2010

(54) CRYSTALLINE FORMS OF SITAGLIPTIN PHOSPHATE

(76) Inventors: Nurit Perlman, Kfar Saba (IL); Revital Ramaty, Ramat-Hasharon (IL); Mili Abramov, Givataim (IL); Nina Finkelstein, Herzliya (IL); Eli Lancry, Modiin (IL); Shay Asis, Rishon Lezion (IL); Ariel Mittelman, Elad (IL)

Correspondence Address: KENYON & KENYON LLP, ONE BROADWAY, NEW YORK, NY, 10004, US

(21) Appl. No.: 12/410,738

(22) Filed: Mar. 25, 2009

Related U.S. Application Data
(60) Provisional application No. 61/154,491, filed on Feb. 23, 2009.
(60) Provisional application No. 61/201,304, filed on Dec. 8, 2008.
(60) Provisional application No. 61/190,868, filed on Sep. 2, 2008.
(60) Provisional application No. 61/092,555, filed on Aug. 28, 2008.
(60) Provisional application No. 61/090,736, filed on Aug. 21, 2008.
(60) Provisional application No. 61/189,128, filed on Aug. 14, 2008.
(60) Provisional application No. 61/070,866, filed on Mar. 25, 2008.
(60) Provisional application No. 61/201,860, filed on Dec. 15, 2008.
(60) Provisional application No. 61/191,933, filed on Sep. 11, 2008.
(60) Provisional application No. 61/091,759, filed on Aug. 26, 2008.
(60) Provisional application No. 61/137,489, filed on Jul. 30, 2008.
(60) Provisional application No. 61/134,598, filed on Jul. 10, 2008.

(51) Int. Cl. C07D 471/08

(52) U.S. Cl. 544/350

(57) ABSTRACT

A Sitagliptin phosphate characterized by data selected from the group consisting of: a powder XRD pattern with peaks at 4.7, 13.5, 17.7, 18.3, and 23.7±0.2 degrees two theta; a powder XRD pattern with peaks at about 4.7, 13.5, and 15.5±0.2 degrees two theta and at least another two peaks selected from the following list: 14.0, 14.4, 18.3, 19.2, 19.5 and 23.7±0.2 degrees two theta; and a powder XRD pattern with peaks at about 13.5, 19.2, and 19.5±0.2 degrees two theta and at least another two peaks selected from the following list: 4.7, 14.0, 15.1, 15.5, 18.3, and 18.7±0.2 degrees two theta; a powder XRD pattern with peaks at about 13.5, 15.5, 19.2, 23.7, and 24.4±0.2 degrees two theta; and a powder XRD pattern with peaks at about 4.65, 13.46, 17.63, 18.30, and 23.66±0.10 degrees two theta, processes for preparing said Sitagliptin crystalline form, and pharmaceutical compositions thereof, are provided.

### RELATED APPLICATIONS

This application claims benefit of U.S. Provisional Patents Application No. 61/154,491, filed Feb. 23, 2009, 61/201,304, filed Dec. 8, 2008, 61/190,868, filed Sep. 2, 2008, 61/092,555, filed Aug. 28, 2008, 61/090,736, filed Aug. 21, 2008, 61/189,128, filed May 14, 2008, and 61/070,866, filed Mar. 25, 2008, the contents of which are incorporated herein in their entirety by reference. This application also claims benefit of U.S. Provisional Patents Application No. 61/201,860, filed Dec. 15, 2008, 61/191,933, filed Sep. 11, 2008, 61/091,759, filed Aug. 26, 2008, 61/137,489, filed Jul. 30, 2008, and 61/134,598, filed Jul. 10, 2008, the contents of which are incorporated herein in their entirety by reference.

### FIELD OF THE INVENTION

The invention encompasses crystalline forms of Sitagliptin phosphate, processes for preparing the crystalline form, and pharmaceutical compositions thereof.

### BACKGROUND OF THE INVENTION

Sitagliptin, (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one, has the following chemical structure:

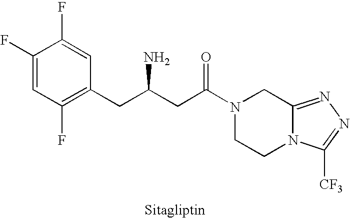

Sitagliptin phosphate is a glucagon-like peptide 1 metabolism modulator, hypoglycemic agent, and dipeptidyl peptidase IV inhibitor. Sitagliptin is currently marketed in its phosphate salt in the United States under the tradename JANUVIA™ in its monohydrate form. JANUVIA™ is indicated to improve glycemic control in patients with type 2 diabetes mellitus.

The following PCT Publications describe the synthesis of Sitagliptin via stereoselective reduction: WO 2004/087650, WO 2004/085661, and WO 2004/085378.

Several crystalline forms of Sitagliptin phosphate are described in the literature. WO 2005/020920 describes crystalline forms I, II, III and ethanol solvate; WO 2005/030127 describes crystalline form IV; WO 2005/003135 describes a monohydrate form, and WO 2006/033848 described the amorphous form.

Polymorphism, the occurrence of different crystal forms, is a property of some molecules and molecular complexes. A single molecule, like Sitagliptin, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Therefore, there is a need for additional crystalline forms of Sitagliptin.

### SUMMARY OF THE INVENTION

The present invention provides a crystalline Sitagliptin phosphate characterized by data selected from the group consisting of: a powder XRD pattern with peaks at 4.7, 13.5, 17.7, 18.3, and 23.7±0.2 degrees two theta; a powder XRD pattern with peaks at about 4.7, 13.5, and 15.5±0.2 degrees two theta and at least another two peaks selected from the following list: 14.0, 14.4, 18.3, 19.2, 19.5 and 23.7±0.2 degrees two theta; a powder XRD pattern with peaks at about 13.5, 19.2, and 19.5±0.2 degrees two theta and at least another two peaks selected from the following list: 4.7, 14.0, 15.1, 15.5, 18.3, and 18.7±0.2 degrees two theta; a powder XRD pattern with peaks at about 13.5, 15.5, 19.2, 23.7, and 24.4±0.2 degrees two theta; and a powder XRD pattern with peaks at about 4.65, 13.46, 17.63, 18.30, and 23.66±0.10 degrees two theta, and processes for preparing thereof.

The present invention also provides a crystalline Form VI of Sitagliptin phosphate characterized by data selected from the group consisting of: a PXRD pattern having peaks at about 13.6, 14.3, 15.6, 16.9, and 19.1±0.2 degrees two theta or peaks at about 17.9, 20.3, 24.8, 26.3, and 28.9±0.2 degrees two theta; a solid-state ¹³C NMR spectrum with signals at about 103.0, 121.5 and 173.2±0.2 ppm; and a solid-state ¹³C NMR spectrum having chemical shifts differences between the signal exhibiting the lowest chemical shift and another in the chemical shift range of 100 to 180 ppm of about 0.0, 18.5 and 70.2±0.1 ppm, wherein, the signal exhibiting the lowest chemical shift in the chemical shift area of 100 to 180 ppm is typically at about 103.0±1 ppm, and processes for preparing thereof.

The present invention further provides processes for the preparation of crystalline Sitagliptin phosphate Form II, Sitagliptin phosphate monohydrate, and amorphous Sitagliptin.

The invention further provides a pharmaceutical formulation comprising the above described Sitagliptin phosphate crystalline forms. This pharmaceutical composition may additionally comprise at least one pharmaceutically acceptable excipient.

The invention further provides a pharmaceutical formulation comprising the above described Sitagliptin phosphate crystalline forms made by the processes of the present invention, and one or more pharmaceutically acceptable excipients.

### BRIEF DESCRIPTION OF THE FIGURES

FIG. 1 shows a powder XRD pattern of a crystalline form of Sitagliptin phosphate, obtained in Example 1.

FIG. 2 shows a powder XRD pattern of a crystalline form of Sitagliptin phosphate, obtained in Example 2.

FIG. 3 shows a powder XRD pattern of a dry crystalline form of Sitagliptin phosphate, obtained in Example 3.

FIG. 4 shows a powder XRD pattern of a crystalline form of Sitagliptin phosphate, obtained in Example 4.

FIG. 5a shows a powder XRD pattern of wet crystalline Form II of Sitagliptin phosphate, obtained in Example 5.

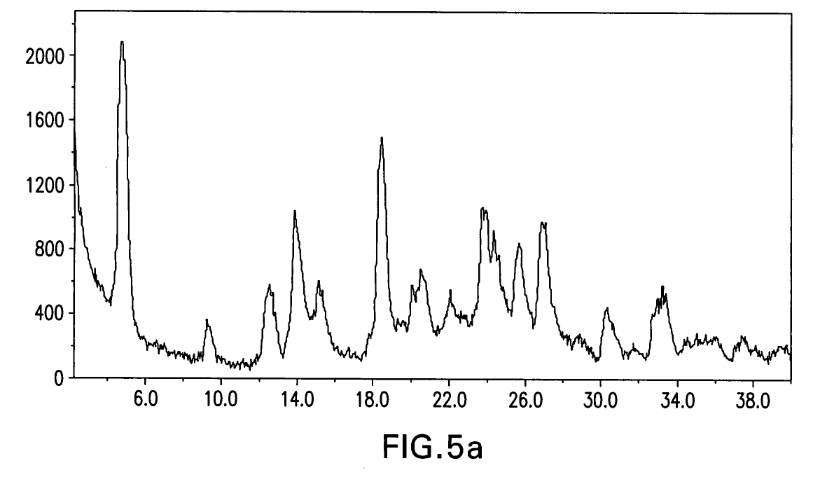

FIG. 5b shows a powder XRD pattern of a dry crystalline form of Sitagliptin phosphate, obtained in Example 5.

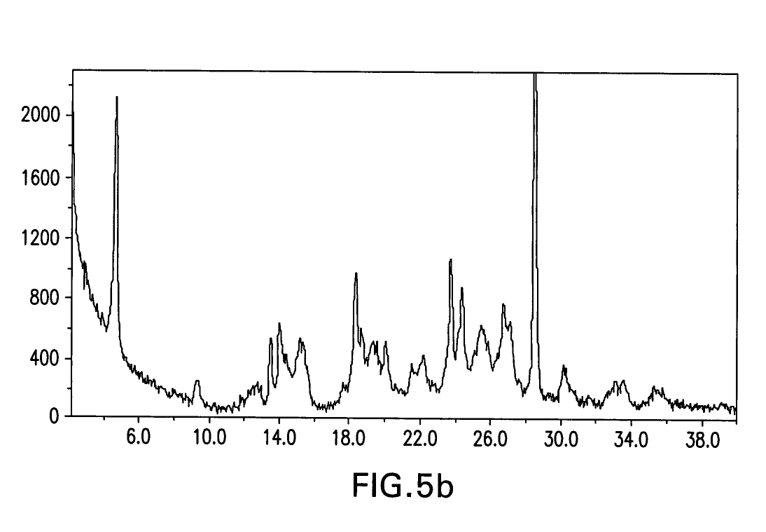

FIG. 6 shows a powder XRD pattern of a crystalline Form II of Sitagliptin phosphate, obtained in Example 33.

FIG. 7 shows a powder XRD pattern of amorphous Sitagliptin phosphate, obtained in Example 63.

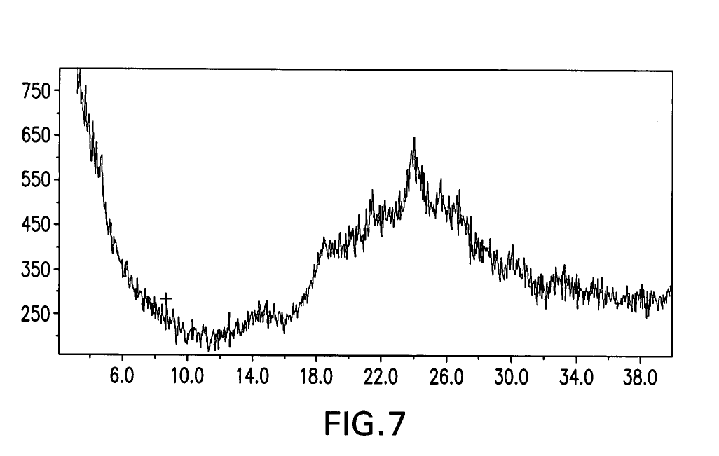

FIG. 8 shows a powder XRD pattern of amorphous Sitagliptin phosphate, obtained in Example 73.

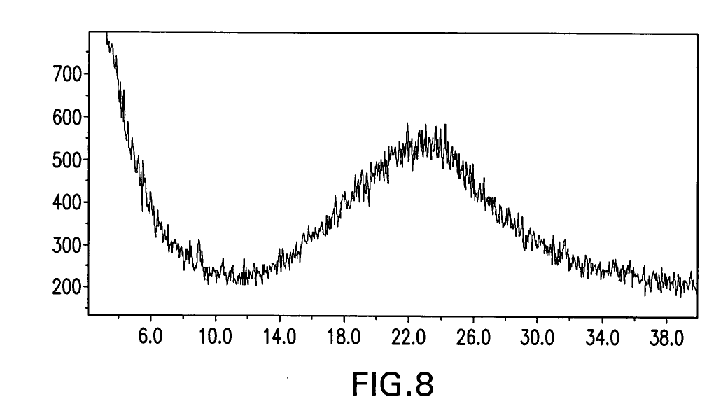

FIG. 9 shows a powder XRD pattern of amorphous Sitagliptin phosphate, obtained in Example 74.

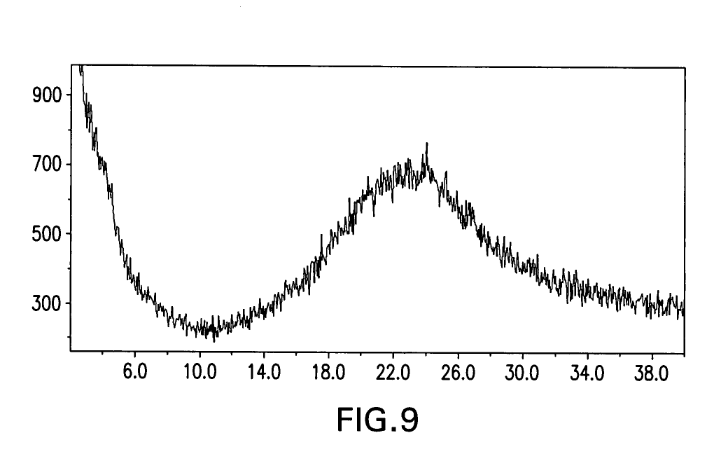

FIG. 10 shows a powder XRD pattern of a crystalline Form II of Sitagliptin phosphate, obtained in Example 78.

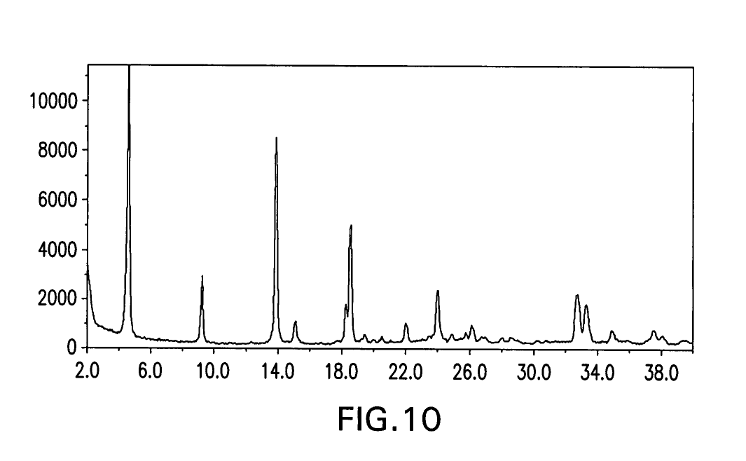

FIG. 11 shows a powder XRD pattern of Sitagliptin phosphate monohydrate, obtained in Example 84.

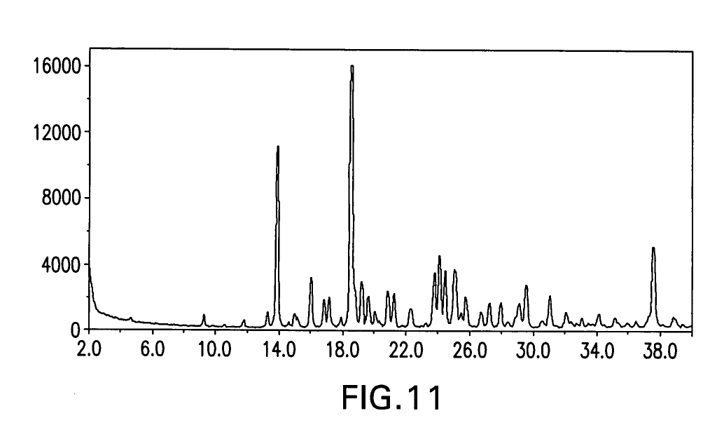

FIG. 12 shows a powder XRD pattern of Sitagliptin phosphate monohydrate, obtained in Example 86.

FIG. 13 shows a powder XRD pattern of Sitagliptin phosphate monohydrate, obtained in Example 87.

FIG. 14a shows a powder XRD pattern of a crystalline form of Sitagliptin phosphate, obtained in Example 88.

FIG. 14b shows a powder XRD pattern of a crystalline form of Sitagliptin phosphate, obtained in Example 88.

FIG. 15 shows a powder XRD pattern of a crystalline form of Sitagliptin phosphate, obtained in Example 92.

FIG. 16 shows a powder XRD pattern of crystalline Form II of Sitagliptin phosphate, obtained in Example 96.

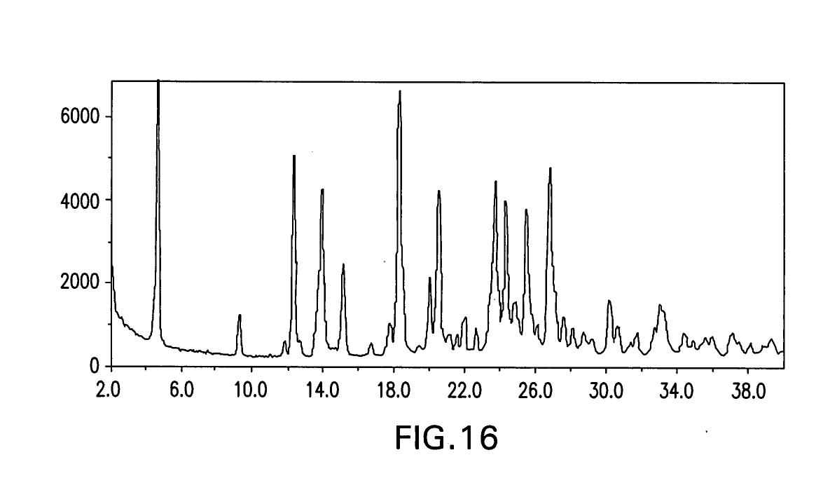

FIG. 17 shows a solid-state ³¹P NMR spectrum of a crystalline form of Sitagliptin phosphate in the (−150)-(150) ppm range.

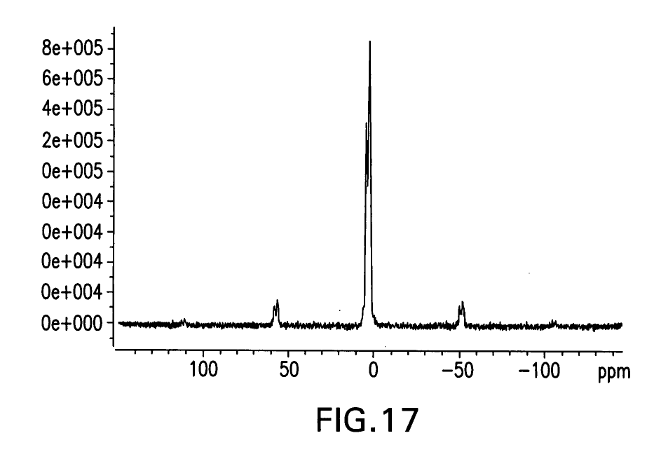

FIG. 18 shows a solid-state ³¹P NMR spectrum of a crystalline form of Sitagliptin phosphate in the (−20)-(20) ppm range.

### DETAILED DESCRIPTION OF THE INVENTION

As used herein, Sitagliptin base Form I refers to crystalline Sitagliptin base characterized by data selected from the group consisting of: a PXRD pattern having any 5 peaks selected from the group consisting of 7.4, 11.5, 16.7, 17.7, 18.9, 24.1, 24.5, 27.0, 28.5 and 28.8±0.2 degrees 2-theta, wherein any combination of peaks selected includes the peak at 7.4±0.2 degrees two theta; a powder XRD pattern with peaks at about 7.4, 16.7, 17.7, 28.5 and 28.8±0.2 degrees 2-theta; a powder XRD pattern with peaks at about 7.4, 11.5, 16.7, 17.7 and 18.9±0.2 degrees 2-theta; a powder XRD pattern with peaks at about 7.4, 11.5, 16.7, 28.5 and 28.8±0.2 degrees 2-theta and a powder XRD pattern with peaks at about 7.4, 24.1, 24.5, 27.0, and 28.8±0.2 degrees 2-theta.

As used herein, Sitagliptin phosphate Form II refers to crystalline Sitagliptin base characterized by a powder XRD pattern with peaks at about 4.7, 9.3, 12.3, 13.9, 15.1, 20.5±0.2 degrees two theta.

As used herein, Sitagliptin phosphate monohydrate refers to crystalline Sitagliptin base characterized by a powder XRD pattern with peaks at about 11. 8, 13.9, 16.0, 18.5, 19.6, 22.5±0.2 degrees two theta.

As used herein, the terms “Sitagliptin phosphate” and “Sitagliptin dihydrophosphate” may be both used to describe Sitagliptin phosphate having a 1:1 ratio of Sitagliptin and phosphate.

As used herein, the term “slurry” refers to a thin mixture of a liquid and a finely divided substance, such as any form of Sitagliptin phosphate. Typically, the solvent is used in an amount that does not result in the full dissolution of the substance.

As used herein, an “antisolvent” refers to a liquid that, when added to a solution of Sitagliptin bas, and phosphoric acid, or a solution of Sitagliptin phosphate in a solvent, induces precipitation of Sitagliptin phosphate.

As used herein, a “wet crystalline form” refers to a polymorph that was not dried using any conventional techniques.

As used herein, a “dry crystalline form” refers to a polymorph that was dried using any conventional techniques. For example, drying at elevated temperature under reduced pressure. Preferably, the crystalline form is dried at about 40° C. to about 60° C., more preferably, between about 45° C. and about 55° C., and, most preferably, about 50° C. Preferably the drying is carried out under reduced pressure (for example less than 1 atmosphere, more preferably, about 10 mbar to about 100 mbar, more preferably, about 10 mbar to about 25 mbar). Preferably the drying takes place over a period of about 8 hours to about 36 hours, more preferably, about 10 hours to about 24 hours, and, most preferably, about 12 hours.

As used herein, the term “room temperature” preferably refers to a temperature of about 20° C. to about 35° C., more preferably, about 25° C. to about 35° C., even more preferably, about 25° C. to about 30° C., and, most preferably, about 25° C.

As used herein, the term “overnight” preferably refers to about 14 hours to about 24 hours, more preferably about 14 hours to about 20 hours, and most preferably about 16 hours.

The present invention provides a crystalline Sitagliptin phosphate characterized by data selected from the group consisting of: a powder XRD pattern with peaks at 4.7, 13.5, 17.7, 18.3, and 23.7±0.2 degrees two theta; a powder XRD pattern with peaks at about 4.7, 13.5, and 15.5±0.2 degrees two theta and at least another two peaks selected from the following list: 14.0, 14.4, 18.3, 19.2, 19.5, and 23.7±0.2 degrees two theta; and a powder XRD pattern with peaks at about 13.5, 19.2, and 19.5±0.2 degrees two theta and at least another two peaks selected from the following list: 4.7, 14.0, 15.1, 15.5, 18.3, and 18.7±0.2 degrees two theta; a powder XRD pattern with peaks at about 13.5, 15.5, 19.2, 23.7, and 24.4±0.2 degrees two theta; and a powder XRD pattern with peaks at about 4.65, 13.46, 17.63, 18.30, and 23.66±0.10 degrees two theta.

In another embodiment, the crystalline form of Sitagliptin phosphate is characterized by a powder XRD pattern with peaks at about 4.7, 13.5, 17.7, 18.3, and 23.7±0.2 degrees two theta.

In another embodiment, the crystalline form of Sitagliptin phosphate is characterized by a powder XRD pattern with peaks at about 13.5, 15.5, 19.2, 23.7, and 24.4±0.1 degrees two theta.

In another embodiment, the crystalline form of Sitagliptin phosphate is further characterized by a powder XRD pattern with peaks at 4.7, 13.5, 17.7, 18.3, and 23.7±0.2 degrees two theta.

In another embodiment, the crystalline form of Sitagliptin phosphate is characterized by a powder XRD pattern with peaks at about 4.65, 13.46, 17.63, 18.30, and 23.66±0.10 degrees two theta.

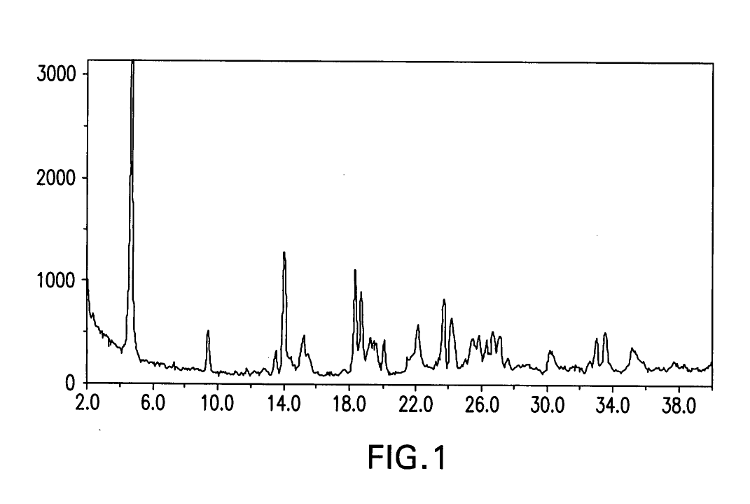

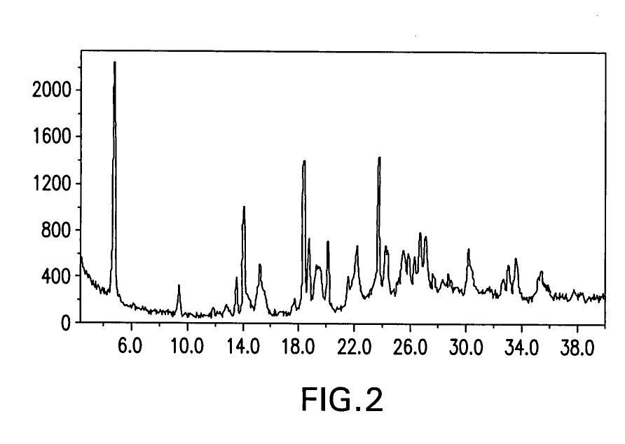

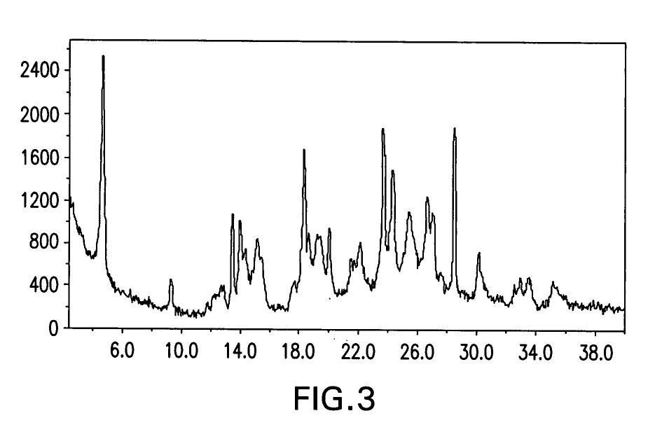

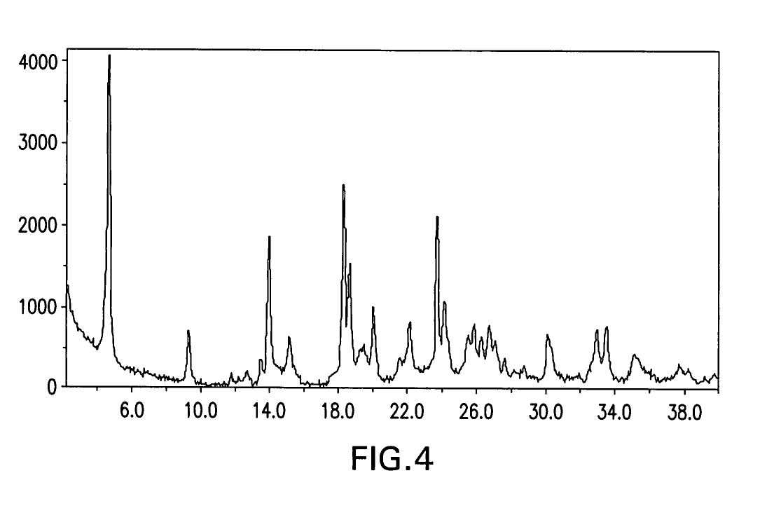

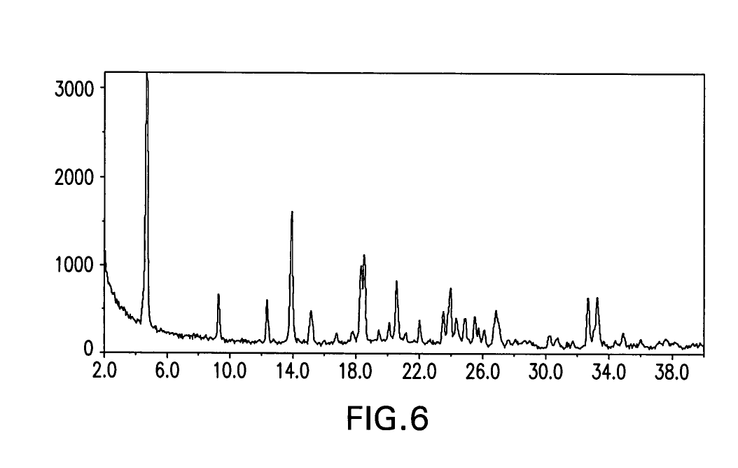

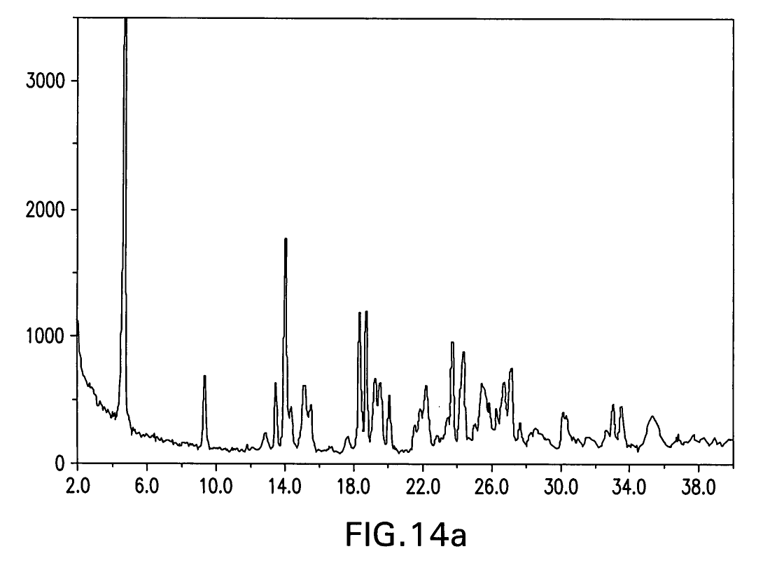

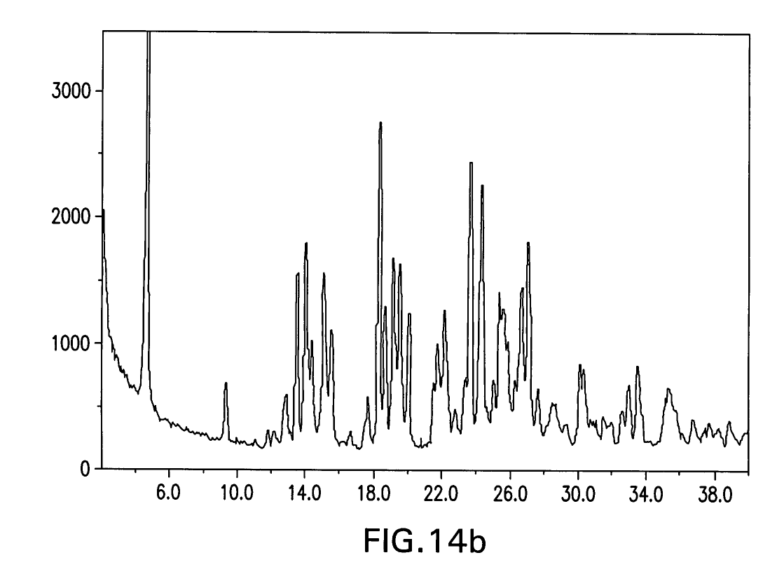

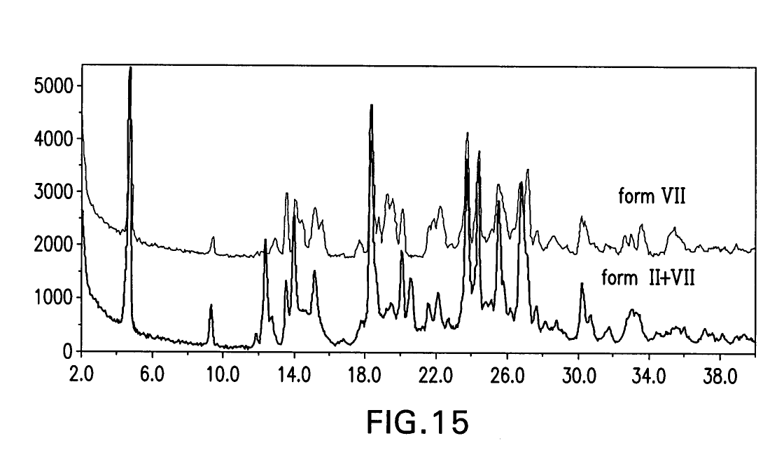

The crystalline form of Sitagliptin phosphate is also characterized by the XRD diffractograms shown in FIGS. 1 to 4, 6, 14, and 15.

The crystalline form of Sitagliptin phosphate, which is characterized by a powder XRD pattern with peaks at 4.7, 13.5, 17.7, 18.3, and 23.7±0.2 degrees two theta, is substantially free of the (S)-enantiomer of Sitagliptin phosphate. By “substantially free” is meant 10% (w/w) or less, more preferably 5% (w/w) or less, most preferably 2% (w/w) or less, particularly 1% (w/w) or less, more particularly 0.5% (w/w) or less, and most particularly 0.2% (w/w) or less.

The crystalline form of Sitagliptin phosphate, which is characterized by a powder XRD pattern with peaks at 4.7, 13.5, 17.7, 18.3, and 23.7±0.2 degrees two theta, is also substantially free of any other polymorph forms. By “substantially free” is meant 20% (w/w) or less, preferably 10% (w/w) or less, more preferably 5% (w/w) or less, most preferably 2% (w/w) or less, particularly 1% (w/w) or less, more particularly 0.5% (w/w) or less, and most particularly 0.2% (w/w) or less.

In another embodiment, the present invention encompasses a crystalline Form VI of Sitagliptin phosphate characterized by data selected from the group consisting of: a PXRD pattern having peaks at about 13.6, 14.3, 15.6, 16.9, and 19.1±0.2 degrees two theta or peaks at about 17.9, 20.3, 24.8, 26.3, and 28.9±0.2 degrees two theta; a solid-state ¹³C NMR spectrum with signals at about 103.0, 121.5 and 173.2±0.2 ppm; and a solid-state ¹³C NMR spectrum having chemical shifts differences between the signal exhibiting the lowest chemical shift and another in the chemical shift range of 100 to 180 ppm of about 0.0, 18.5 and 70.2±0.1 ppm, wherein, the signal exhibiting the lowest chemical shift in the chemical shift area of 100 to 180 ppm is typically at about 103.0±1 ppm.

Form VI is preferably obtained as a mixture of from about 50% to about 85% of the enantiomer R, and from about 15% to about 50% of the enantiomer S, more preferably from about 50% to about 80% of the enantiomer R, and from about 20% to about 50% of the enantiomer S, more preferably about 60% to about 80% of the enantiomer R, and from about 20% to about 40% of the enantiomer S. In one specific embodiment, Form VI is obtained as a mixture of about 77% of the enantiomer R and about 23% of the enantiomer S.

In another example, the crystalline form, characterized by a powder XRD pattern with peaks at 4.7, 13.5, 17.7, 18.3, and 23.7±0.2 degrees two theta, is obtained in a process comprising combining Sitagliptin base and phosphoric acid and a solvent selected from the group consisting of ethyl acetate, dioxane, methyl isobutyl ketone, isobutyl acetate, butyl acetate, a mixture of acetonitrile and toluene, or a mixture of tetrahydrofuran and water, forming a slurry; and obtaining the crystalline form of Sitagliptin phosphate. The obtained slurry is formed either by adding the phosphoric acid to a slurry of the Sitagliptin base in the organic solvent, or by adding the Sitagliptin base into a slurry of the phosphoric acid in the organic solvent.

Preferably the acetonitrile:toluene and the tetrahydrofuran:water ratio is about 1:1 to about 1:15, and most preferably about 3:10. Preferably, the solution is heated to a temperature of about 45° C. to about 80° C., more preferably about 50° C. to about 70° C., preferably, for about 10 minutes to about 5 hours, more preferably for about 20 minutes to about 3 hours. To promote precipitation, the solution can be cooled. Preferably, solution is gradually cooled to a temperature of about room temperature, and stirred until a precipitate is obtained. Preferably, the solution is stirred overnight. The precipitate is further recovered by any conventional method known in the art, for example by filtration. The precipitate may be further dried at about 40° C. to about 60° C., preferably between about 45° C. and about 55° C., most preferably about 50° C. Preferably the drying is carried out under reduced pressure (for example less than 1 atmosphere, more preferably, about 10 mbar to about 100 mbar, more preferably, about 10 mbar to about 25 mbar). Preferably the drying takes place over a period of about 8 hours to about 36 hours, more preferably, about 10 hours to about 24 hours, and, most preferably, about 12 hours.

In another embodiment, the present invention encompasses another process for preparing the crystalline form of Sitagliptin phosphate, which is characterized by a powder XRD pattern with peaks at 4.7, 13.5, 17.7, 18.3, and 23.7±0.2 degrees two theta, comprising combining Sitagliptin base and phosphoric acid and a mixture of a first organic solvent and a second organic solvent selected from the group consisting of acetone:n-hexane, acetone:n-heptane, acetone:cyclopentyl methyl ether, acetone:dibutyl ether, acetone:isopropylacetate, dimethylsulfoxide:methyl isobutyl ketone, and dimethylsulfoxide:methyl tert butyl ether; forming a mixture, and crystallizing Sitagliptin phosphate from the mixture. Where acetone:cyclopentyl methyl ether, acetone:isopropylacetate, and dimethylsulfoxide:methyl tert butyl ether are used, the obtained precipitate is further dried.

Preferably, the first organic solvent and the second organic solvent ratio is about 1:1 to about 1:15, and most preferably about 3:10. Alternatively, Sitagliptin phosphate can be used instead of Sitagliptin base and phosphoric acid.

Preferably, the mixture is heated to a temperature of about 45° to about 80° C., preferably to about 70° C., preferably for about an hour to about 4 hours, more preferably, for about 2 hours. To promote precipitation, the solution can be cooled. Preferably, mixture is gradually cooled to about room temperature with stirring overnight to allow the product to precipitate out. The precipitate is further recovered by any conventional method known in the art, for example by filtration.

The obtained mixture is formed either by adding the phosphoric acid to a mixture of the Sitagliptin base in the organic solvent, or by adding the Sitagliptin base into a mixture of the phosphoric acid in the organic solvent.

In another embodiment, the present invention encompasses another process for preparing the crystalline form of Sitagliptin phosphate of the present invention, comprising drying wet Form II.

Preferably, wet Form II is dried at about 40° to about 100° C., more preferably, at about 40° C. to about 60° C., even more preferably, between about 45° C. and about 55° C., and, most preferably at about 50° C. Preferably, the drying is carried out under reduced pressure (for example less than 1 atmosphere, more preferably, about 10 mbar to about 100 mbar, and, most preferably, about 10 mbar to about 25 mbar). Preferably, the drying takes place over a period of about 8 hours to about 36 hours, more preferably, about 10 hours to about 24 hours, and, most preferably, about 12 hours.

Wet Form II can be prepared by any method known in the art.

For example, wet Form II is obtained in a process comprising combining Sitagliptin base and phosphoric acid and an organic solvent selected from the group consisting of dimethyl carbonate, tetrahydrofuran, propylene glycol methyl ether, methyl ethyl ketone, ethanol, methyl acetate, dimethylformamide, diethyl carbonate, n-butanol, 1-propanol, toluene, isobutyl acetate, isopropyl acetate, isopropanol, a mixture of acetonitrile and n-butanol, acetonitrile, dimethyl carbonate, forming a slurry; and obtaining Sitagliptin phosphate Form II.

Preferably, the slurry is maintained at a temperature of about room temperature to about 70° C. Preferably, the slurry is heated to a temperature of about 50° C. to about 70° C., preferably, for about 10 minutes to about 5 hours, and, more preferably, for about 10 minutes to about 3 hours. Preferably, when the slurry is heated, it is gradually cooled to about 0° C. to about room temperature, more preferably about 10° C. to about room temperature, and, most preferably, about room temperature, and, preferably, stirred overnight to allow the product to precipitate out. The precipitate is further recovered by any conventional method known in the art, for example by filtration.

The obtained slurry is formed either by adding the phosphoric acid to a slurry of the Sitagliptin base in the organic solvent, or by adding the Sitagliptin base into a slurry of the phosphoric acid in the organic solvent.

In another example, wet Form II is prepared in a process comprising combining Sitagliptin base and phosphoric acid and a mixture of a first organic solvent and a second organic solvent selected from the group consisting of acetone:isopropylacetate, acetone:cyclohexane, acetone:isobutyl acetate, acetonitrile:n-butanol, and acetone:n-butanol, forming a mixture; crystallizing Sitagliptin phosphate from the mixture; and obtaining Sitagliptin phosphate Form II.

Preferably, the first organic solvent and the second organic solvent ratio is about 1:1 to about 1:15, and most preferably about 3:10.

Preferably, the mixture is heated to a temperature of about 45° C. to about 70° C., preferably to about 70° C., preferably for about an hour to about 4 hours, more preferably, for about 2 hours. To promote precipitation, the solution can be cooled. Preferably, the mixture is gradually cooled to about 0° C. to about room temperature, more preferably, about 10° C. to about room temperature, and, most preferably, to about room temperature with stirring overnight to allow the product to precipitate out. The precipitate is recovered by any conventional method known in the art, for example by filtration.

The obtained mixture is formed either by adding the phosphoric acid to a mixture of the Sitagliptin base in the organic solvent, or by adding the Sitagliptin base into a mixture of the phosphoric acid in the organic solvent.

In one specific embodiment, the crystalline form of Sitagliptin phosphate, characterized by a powder XRD pattern with peaks at 4.7, 13.5, 17.7, 18.3, and 23.7±0.2 degrees two theta, is prepared in a process comprising drying wet Form II, wherein the wet Form II comprises a solvent selected from the group consisting of methyl isobutyl ketone, dimethyl carbonate, tetrahydrofuran, acetonitrile, propylene glycol methyl ether, methanol, n-butanol, 1-propanol, toluene, isobutyl acetate, isopropyl acetate, butyl acetate, isopropanol, dimethyl carbonate, n-hexane, acetone, cyclohexane, isobutyl acetate, and mixtures thereof.

In another embodiment, the present invention encompasses a process for preparing crystalline Sitagliptin phosphate, characterized by a powder XRD pattern with peaks at 4.7, 13.5, 17.7, 18.3, and 23.7±0.2 degrees two theta, comprising heating a mixture of Sitagliptin phosphate Form II and the crystalline form characterized by a powder XRD pattern with peaks at 4.7, 13.5, 17.7, 18.3, and 23.7±0.2 degrees two theta to a temperature of about 40° C. to about 100° C., and, more preferably, about 40° C. to about 60° C., under reduced pressure (for example less than 1 atmosphere, more preferably, about 10 mbar to about 100 mbar, and, most preferably, about 10 mbar to about 25 mbar). Preferably, the mixture of Sitagliptin phosphate Form II and crystalline Sitagliptin phosphate, characterized by a powder XRD pattern with peaks at 4.7, 13.5, 17.7, 18.3, and 23.7±0.2 degrees two theta, is heated over a period of about 8 hours to about 36 hours, more preferably, about 10 hours to about 24 hours, and, most preferably, about 12 hours.

In another embodiment, the present invention encompasses another process for preparing crystalline Sitagliptin phosphate, characterized by a powder XRD pattern with peaks at 4.7, 13.5, 17.7, 18.3, and 23.7±0.2 degrees two theta, comprising drying a mixture of Sitagliptin phosphate Form II and the crystalline Sitagliptin phosphate characterized by a powder XRD pattern with peaks at 4.7, 13.5, 17.7, 18.3, and 23.7±0.2 degrees two theta, in a fluidized bed dryer at a temperature of about 30° C. to about 60° C., more preferably about 35° C. to about 50° C.

In another embodiment, the present invention encompasses a crystalline form of Sitagliptin phosphate, characterized by data selected from the group consisting of: a powder XRD pattern with peaks at 4.7, 13.5, 17.7, 18.3, and 23.7±0.2 degrees two theta; a powder XRD pattern with peaks at about 4.7, 13.5, and 15.5±0.2 degrees two theta and at least another two peaks selected from the following list: 14.0, 14.4, 18.3, 19.2, 19.5 and 23.7±0.2 degrees two theta; and a powder XRD pattern with peaks at about 13.5, 19.2, and 19.5±0.2 degrees two theta and at least another two peaks selected from the following list: 4.7, 14.0, 15.1, 15.5, 18.3, and 18.7±0.2 degrees two theta; a powder XRD pattern with peaks at about 13.5, 15.5, 19.2, 23.7, and 24.4±0.2 degrees two theta; and a powder XRD pattern with peaks at about 4.65, 13.46, 17.63, 18.30, and 23.66±0.10 degrees two theta, made by the processes described above.

In another embodiment, the present invention encompasses a process for preparing Form II comprising providing a slurry of Sitagliptin phosphate characterized by a powder XRD pattern with peaks at 4.7, 13.5, 17.7, 18.3, and 23.7±0.2 degrees two theta, and a solvent selected from the group consisting of acetonitrile, methanol, ethanol, 1-propanol, isopropanol, acetone, tetrahydrofuran, n-butanol, iso-butanol, toluene, propylene glycol, propylene glycol methyl ether, chloroform, diethyl carbonate, dimethylformamide, or mixtures of dimethylformamide with methyl isobutyl ketone, or n-butanol; heating the slurry; and recovering the obtained Form II.

Preferably, the mixture is heated at a temperature of about 50° C. to about 80° C., more preferably, about 60° C. to about 75° C., even more preferably, about 65° C. to about 75° C., and, most preferably, about 70° C. The mixture is preferably stirred at this temperature for about 5 minutes to about 5 hours, and, more preferably, about 10 minutes to about 3 hours. Preferably, the mixture is gradually cooled to about 0° C. to about room temperature, more preferably about 10° C. to about room temperature, and, most preferably, to about room temperature. The mixture is stirred at this temperature overnight. The precipitate is further recovered by any conventional method known in the art, for example by filtration.

In another the present invention encompasses another process for preparing Form II comprising combining Sitagliptin base and phosphoric acid and an organic solvent selected from the group consisting of dimethyl carbonate, tetrahydrofuran, propylene glycol methyl ether, methyl ethyl ketone, ethanol, methyl acetate, dimethylformamide, diethyl carbonate, n-butanol, 1-propanol, toluene, isobutyl acetate, isopropyl acetate, isopropanol, a mixture of acetonitrile and n-butanol, acetonitrile, dimethyl carbonate, and a mixture of dimethyl carbonate and n-hexane, forming a slurry; and obtaining Form II.

Preferably, the slurry is maintained at a temperature of about room temperature to about 70° C. More preferably, the slurry is heated to a temperature of about 50° C. to about 70° C., preferably for about 10 minutes to about 5 hours, more preferably for about 10 minutes to about 3 hours. Preferably, when the slurry is heated, it is gradually cooled to a temperature of about 0° C. to about room temperature, more preferably about 10° C. to about room temperature, and most preferably to about room temperature and stirring, preferably overnight to allow the product to precipitate out. The precipitate is further recovered by any conventional method known in the art, for example by filtration.

The obtained slurry is formed either by adding the phosphoric acid to a slurry of the Sitagliptin base in the organic solvent, or by adding the Sitagliptin base into a slurry of the phosphoric acid in the organic solvent.

In another embodiment, the present invention encompasses another process for preparing Form II comprising combining Sitagliptin base and phosphoric acid and a mixture of a first organic solvent and a second organic solvent selected from the group consisting of acetone:isopropylacetate, acetone:cyclohexane, acetone:isobutyl acetate, acetonitrile:n-butanol, and acetone:n-butanol, forming a mixture; crystallizing Sitagliptin phosphate from the mixture; and recovering Sitagliptin phosphate Form II.

Preferably, the first organic solvent and the second organic solvent ratio is about 1:1 to about 1:15, and most preferably about 3:10.

Preferably, the mixture is heated to a temperature of about 45° C. to about 70° C., preferably to about 70° C., preferably for about an hour to about 4 hours, more preferably, for about 2 hours. To promote precipitation, the solution can be cooled. Preferably, mixture is gradually cooled to about 0° C. to about room temperature, more preferably, about 10° C. to about room temperature, and, most preferably, to about room temperature and stirring overnight to allow the product to precipitate out. The precipitate is recovered by any conventional method known in the art, for example by filtration.

The obtained mixture is formed either by adding the phosphoric acid to a mixture of the Sitagliptin base in the organic solvent, or by adding the Sitagliptin base into a mixture of the phosphoric acid in the organic solvent.

In another embodiment, the present invention encompasses another process for preparing Sitagliptin phosphate Form II, comprising dissolving Sitagliptin phosphate in dimethylsulfoxide; adding an antisolvent selected from the group consisting of iso-butanol, acetonitrile, diethyl ether, diethyl carbonate, and tert-butyl ether; and recovering Sitagliptin phosphate Form II.

Preferably, the solvent/antisolvent ratio is about 1:1 to about 1:20, and most preferably about 3:10.

Preferably, the starting Sitagliptin phosphate is crystalline Sitagliptin phosphate characterized by a powder XRD pattern with peaks at 4.7, 13.5, 17.7, 18.3, and 23.7±0.2 degrees two theta.

In order to promote precipitation, the mixture may be cooled to about 0° C. to about 20° C., preferably, for about 2 hours to about 24 hours.

In another embodiment, the present invention encompasses another process for preparing Sitagliptin phosphate Form II, comprising granulating Sitagliptin phosphate in the presence of isopropanol. Preferably, the starting Sitagliptin phosphate is crystalline Sitagliptin phosphate characterized by a powder XRD pattern with peaks at 4.7, 13.5, 17.7, 18.3, and 23.7±0.2 degrees two theta.

The term “granulation” broadly refers to a process comprising mixing the solid with a minimal amount of solvent, and stirring the mixture at about room temperature for the time needed to cause the desired transformation. A mechanical stirrer can be used in the process. Typically, about 0.1 to about 0.2 ml of solvent is used per 1 gram of compound. Preferably, the mixture is granulated using a rotary evaporator.

In another embodiment, the present invention encompasses a process for preparing Form II, comprising exposing Sitagliptin phosphate characterized by a powder XRD pattern with peaks at 4.7, 13.5, 17.7, 18.3, and 23.7±0.2 degrees two theta to a C₁-C₄ alcohol, where the alcohol is preferably selected from the group consisting of ethanol, methanol, and isopropanol.

In another embodiment, the present invention encompasses a process for preparing the crystalline form VI of Sitagliptin phosphate of the present invention, comprising providing a slurry of Sitagliptin phosphate, and an organic solvent selected from the group consisting of acetonitrile (ACN), and C₁-C₄ alcohols, most preferably isopropanol; heating the slurry; cooling the resulting mixture; and recovering the obtained Form VI of Sitagliptin phosphate. Optionally, Sitagliptin phosphate can be formed in situ starting from Sitagliptin base and phosphoric acid. The Sitagliptin base or the Sitagliptin phosphate are introduced as a mixture of from about 50% to about 85% of the enantiomer R, and from about 15% to about 50% of the enantiomer S, more preferably from about 50% to about 80% of the enantiomer R, and from about 20% to about 50% of the enantiomer S, more preferably about 60% to about 80% of the enantiomer R, and from about 20% to about 40% of the enantiomer S.

Preferably, from about 10 ml to about 70 ml of acetonitrile, and, more preferably, about 25 ml to about 60 ml are used per gram of the Sitagliptin phosphate. Preferably, from about 2 ml to about 12 ml, and, more preferably, about 4 ml to about 10 ml of the organic solvent are used per gram of the Sitagliptin.

Preferably, the Sitagliptin or the Sitagliptin salt, which is combined with the ACN, is amorphous.

The obtained slurry is preferably heated to a temperature of about 40 to about reflux, more preferably, the slurry is heated to about 60 to about reflux, and, most preferably, the slurry is heated to about reflux. To induce precipitation, the slurry is then cooled to about 0° C. to about room temperature, more preferably to about 0° C. to about 4° C., and preferably maintained for about 1 day to about 5 days, and, more preferably, for about 3 days, to induce precipitation.

When phosphoric acid is introduced into a mixture of Sitagliptin and the organic solvent, preferably, it is added in a dropwise manner. Preferably, the acid is added to a heated solution or slurry of the Sitagliptin and the organic solvent, where the heated solution or slurry is at a temperature of about 40° C. to about 65° C., and, more preferably about 45° C. to about 60° C.

Preferably, the chemical purity of the obtained Form VI is more than 99.5%, and, more preferably, more than 99.9%.

In another embodiment, the present invention encompasses another process for preparing amorphous Sitagliptin phosphate, comprising dissolving Sitagliptin phosphate in dimethylsulfoxide; and adding an antisolvent selected form a group consisting of methyl tert-butyl ether, and tetrahydrofuran to obtain amorphous Sitagliptin phosphate.

The mixture is maintained at a temperature of about 0° C. for about 2 hours to induce precipitating.

In another embodiment, the present invention encompasses another process for preparing amorphous Sitagliptin phosphate comprising combining Sitagliptin base and phosphoric acid and an organic solvent selected from the group consisting of diethyl carbonate, dimethyl carbonate, and a mixture of cyclohexanone and methyl tert-butyl ether, forming a slurry; and recovering the precipitate from the mixture.

Preferably, the mixture is maintained at a temperature of about 15° C. to about 70° C., preferably about 20° C. to about 50° C. for about 10 minutes to about 7 days, more preferably for about 10 minutes to about an hour.

The obtained slurry is formed either by adding the phosphoric acid to a slurry of the Sitagliptin base in the organic solvent, or by adding the Sitagliptin base into a slurry of the phosphoric acid in the organic solvent.

In another embodiment, the present invention encompasses a process to obtain Sitagliptin phosphate monohydrate comprising heating a mixture of Sitagliptin phosphate with water and an organic solvent selected from a group consisting of methyl tert-butyl ether and acetonitrile; and recovering the precipitate. Alternatively, a mixture of Sitagliptin base and phosphoric acid can be introduced instead of Sitagliptin phosphate.

Preferably, the mixture is heated to about 50° C. to about 80° C., more preferably 60° C. to about 70° C., and then cooled to about 0° C. to about 25° C. Recovering the product may be carried out via any known method in the art, for example by filtration or evaporation.

The invention further provides a pharmaceutical formulation comprising the above described Sitagliptin phosphate crystalline forms. This pharmaceutical composition may additionally comprise at least one pharmaceutically acceptable excipient.

The invention further provides a pharmaceutical formulation comprising the above described Sitagliptin phosphate crystalline forms made by the processes of the present invention, and one or more pharmaceutically acceptable excipients. The compositions of the invention include powders, granulates, aggregates and other solid compositions comprising the present invention form of Sitagliptin solid crystalline.

The present invention also provides methods of treating type 2 diabetes mellitus in a patient, preferably a human, by administrating to the patient a pharmaceutical composition comprising Sitagliptin phosphate crystalline form as described herein. Preferably, the pharmaceutical composition comprises a therapeutically effective amount of Sitagliptin phosphate crystalline form.

The present invention also provides the use of the above mentioned Sitagliptin phosphate crystalline forms, for the manufacture of a pharmaceutical composition for the treatment of type 2 diabetes mellitus.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### Examples

### X-Ray Power Diffraction:

X-Ray powder diffraction data was obtained by using methods known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2-40 degrees two-theta; scan mode: continuous scan; step size: 0.05 deg.; and a rate of 3 deg/min. All peak positions are within ±0.2 degrees two theta.

The PXRD peaks positions are calibrated using silicon powder as internal standard in an admixture with the sample measured. The position of the silicon (111) peak was corrected to be 28.45 degrees two theta. The positions of Sitagliptin phosphate form peaks were corrected respectively. (No correction was performed on the presented diffractograms in the figures).

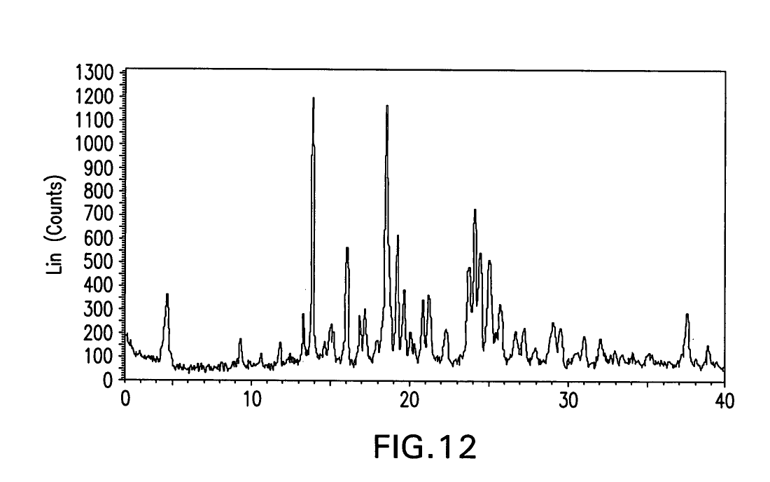

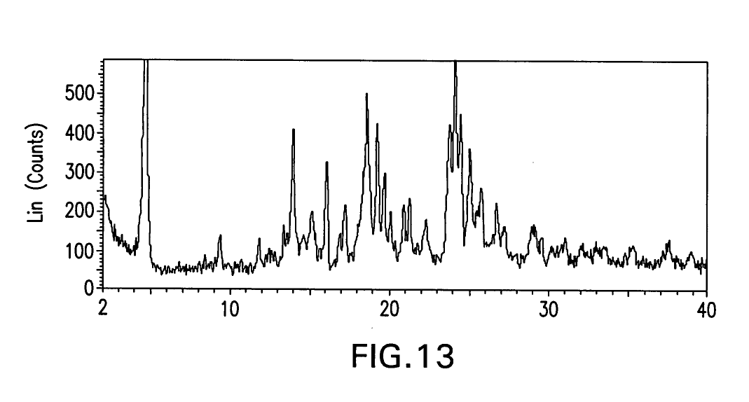

FIGS. 12 and 13 were obtained by using methods known in the art using a Bruker X-Ray powder diffractometer model D8 advance equipped with lynxEye. Scan range: 2-40°. Step size: 0.05°. Time per step: 5.2 seconds.

### NMR Parameters

³¹P NMR at 202 MHz using Bruker Avance II+ 500
SB probe using 4 mm rotors
Magic angle was set using KBr
Homogeneity of magnetic field checked using adamantane
Parameters for Cross polarization optimized using glycine
Spectral reference set according to Ammonium Dihydrogeno Phosphate as external standard (0.00 ppm for signal)

### Scanning Parameters:

Magic Angle Spinning Rate: 11 kHz
Pulse Program: cp with tppm15 during decoupling
Delay time: 25 s

STG (Sitagliptin) base form I can be obtained according to the procedures described in PCT application No. PCT/US08/01317.

### Example 1

STG (Sitagliptin) base form I (100 mg) was dissolved in ethyl acetate (500 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain wet STG phosphate crystalline form characterized by a powder XRD pattern with peaks at 4.7, 13.5, 17.7, 18.3, and 23.7±0.2 degrees two theta.

### Example 2

STG base form I (100 mg) was dissolved in tetrahydrofuran:water 2:1 (300 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain STG phosphate crystalline form characterized by a powder XRD pattern with peaks at 4.7, 13.5, 17.7, 18.3, and 23.7±0.2 degrees two theta.

### Example 3

STG base form I (100 mg) was partially dissolved in methyl isobutyl ketone (1000 μL) at 25° C. Phosphoric acid (85%, 17 μL, 1 eq) was then added and the mixture was heated to 70° C., stirred at 70° C. for 1.5 hours, then cooled gradually to 25° C. and stirred at 25° C. for 16 hours. The product was isolated by vacuum filtration to obtain wet STG phosphate crystalline form characterized by a powder XRD pattern with peaks at 4.7, 13.5, 17.7, 18.3, and 23.7±0.2 degrees two theta. The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 4

STG base form I (100 mg) was partially dissolved in dioxane (1000 μL) at 25° C. Phosphoric acid (85%, 17 μL, 1 eq) was then added and the mixture was heated to 70° C., stirred at 70° C. for 1.5 hours, then cooled gradually to 25° C. and stirred at 25° C. for 16 hours. The product was isolated by vacuum filtration to obtain wet STG phosphate crystalline form characterized by a powder XRD pattern with peaks at 4.7, 13.5, 17.7, 18.3, and 23.7±0.2 degrees two theta.

### Example 5

STG base form I (100 mg) was partially dissolved in dimethyl carbonate (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form II

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 6

STG base form I (100 mg) was dissolved in acetone (100 μL) at 25° C. Then, n-Hexane was added (500 μL) at 25° C. Two phases were formed. Phosphoric acid (85%, 17 μL, 1 eq) was then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form characterized by a powder XRD pattern with peaks at 4.7, 13.5, 17.7, 18.3, and 23.7±0.2 degrees two theta.

### Example 7

STG base form I (100 mg) was dissolved in acetone (100 μL) at 25° C. Phosphoric acid (85%, 17 μL, 1 eq) and n-Hexane (500 μL) were then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form characterized by a powder XRD pattern with peaks at 4.7, 13.5, 17.7, 18.3, and 23.7±0.2 degrees two theta.

### Example 8

STG base form I (100 mg) was dissolved in acetone (100 μL) at 25° C. Phosphoric acid (85%, 17 μL, 1 eq) and n-Heptane (500 μL) were then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form characterized by a powder XRD pattern with peaks at 4.7, 13.5, 17.7, 18.3, and 23.7±0.2 degrees two theta.

### Example 9

STG base form I (100 mg) was dissolved in acetone (100 μL) at 25° C. Phosphoric acid (85%, 17 μL, 1 eq) and cyclopentyl methyl ether (1000 μL) were then added and the mixture was heated to 70° C., stirred at 70° C. for 2 hours, then cooled gradually to 25° C. and stirred at 25° C. for 16 hours.

The product was isolated by vacuum filtration to obtain a mixture of wet STG phosphate crystalline form characterized by a powder XRD pattern with peaks at 4.7, 13.5, 17.7, 18.3, and 23.7±0.2 degrees two theta and form II. The sample was dried in vacuum oven at 50° C. 24 hours to obtain STG phosphate crystalline form characterized by a powder XRD pattern with peaks at 4.7, 13.5, 17.7, 18.3, and 23.7±0.2 degrees two theta.

### Example 10

STG base form I (100 mg) was dissolved in acetone (300 μL) at 25° C. Phosphoric acid (85%, 17 μL, 1 eq) and dibutyl ether (1000 μL) were then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form characterized by a powder XRD pattern with peaks at 4.7, 13.5, 17.7, 18.3, and 23.7±0.2 degrees two theta.

### Example 11

STG base form I (100 mg) was dissolved in methyl ethyl ketone (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain wet STG phosphate crystalline form II.

### Example 12

STG base form I (100 mg) was dissolved in acetone (300 μL) at 25° C. Phosphoric acid (85%, 17 μL, 1 eq) and cyclohexane (1000 μL) were then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form II.

### Example 13

STG phosphate Form characterized by a powder XRD pattern with peaks at 4.7, 13.5, 17.7, 18.3, and 23.7±0.2 degrees two theta (50 mg) was dissolved in dimethylsulfoxide (0.05 ml) at 25° C. Then iso-Butanol (1 ml) was added at 25° C. The solution formed was slurry (crystallization occurred) and was cooled in ice water bath for 2 hrs.

The product was isolated by vacuum filtration to obtain wet STG phosphate crystalline form II.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 14

STG phosphate Form characterized by a powder XRD pattern with peaks at 4.7, 13.5, 17.7, 18.3, and 23.7±0.2 degrees two theta (50 mg) was dissolved in dimethylsulfoxide (0.05 ml) at 25° C. Then Acetonitrile (1 ml) was added at 25° C. Crystallization occurred and the mixture was cooled in ice water bath for 16 hours.

The product was isolated by vacuum filtration to obtain wet STG phosphate crystalline form II.

### Example 15

STG phosphate Form characterized by a powder XRD pattern with peaks at 4.7, 13.5, 17.7, 18.3, and 23.7±0.2 degrees two theta (50 mg) was dissolved in dimethylsulfoxide (0.05 ml) at 25° C. Then diethyl ether (1 ml) was added at 25° C. The solution formed was a slurry (crystallization occurred) and was cooled in ice water bath for 2 hours.

The product was isolated by vacuum filtration to obtain wet STG phosphate crystalline form II.

### Example 16

STG phosphate Form characterized by a powder XRD pattern with peaks at 4.7, 13.5, 17.7, 18.3, and 23.7±0.2 degrees two theta (50 mg) was dissolved in dimethylsulfoxide (0.05 ml) at 25° C. Then diethyl carbonate (1 ml) was added at 25° C. The solution formed was a slurry (crystallization occurred) and was cooled in ice water bath for 2 hours.

The product was isolated by vacuum filtration to obtain wet STG phosphate crystalline form II.

### Example 17

STG base form I (100 mg) was partially dissolved in tetrahydrofuran (500 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain wet STG phosphate crystalline form II.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 18

STG base form I (100 mg) was dissolved in acetonitrile (500 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours.

The product was isolated by vacuum filtration to obtain a mixture of wet STG phosphate crystalline form characterized by a powder XRD pattern with peaks at 4.7, 13.5, 17.7, 18.3, and 23.7±0.2 degrees two theta and form II.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 19

STG base form I (100 mg) was dissolved in ethanol (500 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form II.

### Example 20

STG base form I (100 mg) was dissolved in methyl acetate (1000 μL) at 25° C. Phosphoric acid (85%, 17 μL, 1 eq) was then added and the mixture was heated to 70° C., stirred at 70° C. for 2.5 hours, then cooled gradually to 25° C. and stirred at 25° C. for 16 hours.

The product was isolated by vacuum filtration to obtain wet STG phosphate crystalline form II.

### Example 21

STG base form I (100 mg) was dissolved in propylene glycol methyl ether (1000 μL) at 25° C. Phosphoric acid (85%, 17 μL, 1 eq) was then added and the mixture was heated to 70° C., stirred at 70° C. for 2.5 hours, then cooled gradually to 25° C. and stirred at 25° C. for 16 hours.

The product was isolated by vacuum filtration to obtain wet STG phosphate crystalline form II.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 22

STG base form I (100 mg) was dissolved in dimethyl formamide (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by evaporation to obtain wet STG phosphate crystalline form II.

### Example 23

STG base form I (100 mg) was dissolved in dimethylsulfoxide (200 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by evaporation, addition of methanol and vacuum filtration to obtain wet STG phosphate crystalline form II.

### Example 24

STG base form I (100 mg) was dissolved in dimethyl formamide (500 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain wet STG phosphate crystalline form II.

### Example 25

STG base form I (100 mg) was dissolved in acetone (100 μL) at 25° C. Phosphoric acid (85%, 17 μL, 1 eq) and iso-butyl acetate (500 μL) were then added and the mixture was heated to 70° C., stirred at 70° C. for 2 hours, then cooled gradually to 25° C. and stirred at 25° C. for 16 hours.

The product was isolated by vacuum filtration to obtain STG phosphate crystalline form II.

### Example 26

STG base form I (100 mg) was dissolved in acetone (100 μL) at 25° C. Phosphoric acid (85%, 17 μL, 1 eq) and iso-propyl acetate (1000 μL) were then added and the mixture was heated to 70° C., stirred at 70° C. for 2 hours, then cooled gradually to 25° C. and stirred at 25° C. for 16 hours.

The product was isolated by vacuum filtration to obtain STG phosphate crystalline form II.

The sample was dried at 50° C. for about 24 hours under reduced pressure to obtain STG phosphate crystalline form characterized by a powder XRD pattern with peaks at 4.7, 13.5, 17.7, 18.3, and 23.7±0.2 degrees two theta.

### Example 27

STG base form I (100 mg) was dissolved in acetone (300 μL) at 25° C. Phosphoric acid (85%, 17 μL, 1 eq) and n-butanol (1000 μL) were then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form II.

### Example 28

STG phosphate (50 mg, crystalline form characterized by a powder XRD pattern with peaks at 4.7, 13.5, 17.7, 18.3, and 23.7±0.2 degrees two theta) was slurried in acetonitrile (1 ml) at 25° C., then heated to 70° C., stirred at 70° C. for 5 hours, cooled gradually to 10° C. and remained at 10° C. for 16 hours.

The product was isolated by vacuum filtration to obtain STG phosphate crystalline form II.

### Example 29

STG phosphate (50 mg, crystalline form characterized by a powder XRD pattern with peaks at 4.7, 13.5, 17.7, 18.3, and 23.7±0.2 degrees two theta) was slurried in methanol (1 ml) at room temperature, then heated to 50° C., stirred at 50° C. for 5 hours, cooled gradually to 10° C. and remained at 10° C. for 16 hours.

The product was isolated by vacuum filtration to obtain STG phosphate crystalline form II.

### Example 30

STG phosphate (50 mg, crystalline form characterized by a powder XRD pattern with peaks at 4.7, 13.5, 17.7, 18.3, and 23.7±0.2 degrees two theta) was slurried in acetone (1 ml) at room temperature, then heated to 50° C., stirred at 50° C. 5 for hours, cooled gradually to 10° C. and remained at 10° C. for 16 hours.

The product was isolated by vacuum filtration to obtain STG phosphate crystalline form II.

### Example 31

STG phosphate (50 mg, crystalline form characterized by a powder XRD pattern with peaks at 4.7, 13.5, 17.7, 18.3, and 23.7±0.2 degrees two theta) was slurried in tetrahydrofuran (1 ml) at room temperature, then heated to 50° C., stirred at 50° C. for 5 hours, cooled gradually to 10° C. and remained at 10° C. for 16 hours.

The product was isolated by vacuum filtration to obtain wet STG phosphate crystalline form II.

### Example 32

STG phosphate (50 mg, crystalline form characterized by a powder XRD pattern with peaks at 4.7, 13.5, 17.7, 18.3, and 23.7±0.2 degrees two theta) was slurried in n-Butanol (1 ml) at room temperature, then heated to 95° C., stirred at that temperature for 5 hours, cooled gradually to 10° C. and remained at 10° C. for 16 hours.

The product was isolated by vacuum filtration to obtain wet STG phosphate crystalline form II.

### Example 33

Sitagliptin dihydrophosphate form characterized by a powder XRD pattern with peaks at 4.7, 13.5, 17.7, 18.3, and 23.7±0.2 degrees two theta (0.03g) was slurried in 0.3 ml n-butanol at 25° C., under magnetic stirring for 24 hours. The product was isolated by filtration. The wet material was analyzed by XRD and found to be Sitagliptin dihydrophosphate Form II.

### Example 34

STG phosphate (50 mg, crystalline form characterized by a powder XRD pattern with peaks at 4.7, 13.5, 17.7, 18.3, and 23.7±0.2 degrees two theta) was slurried in iso-Butanol (1 ml) at room temperature, then heated to 95° C., stirred at that temperature for 5 hours, cooled gradually to 10° C. and remained at 10° C. for 16 hours.

The product was isolated by vacuum filtration to obtain wet STG phosphate crystalline form II.

### Example 35

Sitagliptin dihydrophosphate form characterized by a powder XRD pattern with peaks at 4.7, 13.5, 17.7, 18.3, and 23.7±0.2 degrees two theta (0.05g) was slurried in 1 ml iso-BuOH at 50° C., under magnetic stirring for 3 hours and at 10° C. for 16 hours. The product was isolated by filtration. The wet material was analyzed by XRD and found to be Sitagliptin dihydrophosphate Form II.

### Example 36

STG phosphate (50 mg, crystalline form characterized by a powder XRD pattern with peaks at 4.7, 13.5, 17.7, 18.3, and 23.7±0.2 degrees two theta) was slurried in toluene (1 ml) at room temperature, then heated to 95° C., stirred at that temperature for 5 hours, cooled gradually to 10° C. and remained at 10° C. for 16 hours.

The product was isolated by vacuum filtration to obtain wet STG phosphate crystalline form II.

### Example 37

STG phosphate (50 mg, crystalline form characterized by a powder XRD pattern with peaks at 4.7, 13.5, 17.7, 18.3, and 23.7±0.2 degrees two theta) was slurried in N,N-Dimethyl Formamide (1 ml) at room temperature, then heated to 70° C., stirred at that temperature for 4 hours, cooled gradually to 10° C. and remained at 10° C. for 16 hours. The product was isolated by vacuum filtration to obtain wet STG phosphate crystalline form II.

### Example 38

STG phosphate (50 mg, crystalline form characterized by a powder XRD pattern with peaks at 4.7, 13.5, 17.7, 18.3, and 23.7±0.2 degrees two theta) was slurried in N,N-Dimethyl Formamide (0.5 ml) at room temperature. Then Methyl iso-Butyl Ketone (0.5 ml) was added at room temperature. The solution formed was slurry and stirred for 16 hours.

The product was isolated by vacuum filtration to obtain wet STG phosphate crystalline form II.

### Example 39

STG phosphate (50 mg, crystalline form characterized by a powder XRD pattern with peaks at 4.7, 13.5, 17.7, 18.3, and 23.7±0.2 degrees two theta) was slurried in N,N-Dimethyl Formamide (0.5 ml) at room temperature. Then n-butanol (0.5 ml) was added at room temperature. The solution formed was slurry and stirred for 16 hours. The product was isolated by vacuum filtration to obtain wet STG phosphate crystalline form II.

### Example 40

STG phosphate (50 mg, crystalline form characterized by a powder XRD pattern with peaks at 4.7, 13.5, 17.7, 18.3, and 23.7±0.2 degrees two theta) was slurried in propylene glycol (0.025 ml) at 25° C. for 16 hours.

The product was isolated by vacuum filtration to obtain wet STG phosphate crystalline form II.

### Example 41

STG base Form I (100 mg) was slurried in n-butanol (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form II.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 42

STG base Form I (100 mg) was slurried in iso-propanol (1000 μL) at 25° C. Phosphoric acid (85%, 17 μL, 1 eq) was then added and the mixture was heated to 70° C., stirred at 70° C. for 2.5 hours, then cooled gradually to 25° C. and stirred at 25° C. for 16 hours. The product was isolated by vacuum filtration to obtain wet STG phosphate crystalline form II.

### Example 43

STG base Form I (100 mg) was slurried in 1-propanol (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours.

The product was isolated by vacuum filtration to obtain wet STG phosphate crystalline form II.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 44

Sitagliptin dihydrophosphate form characterized by a powder XRD pattern with peaks at 4.7, 13.5, 17.7, 18.3, and 23.7±0.2 degrees two theta (0.03g) was slurried in 0.3 ml 1-propanol at 25° C., under magnetic stirring for 24 hours. The product was isolated by filtration. The wet material was analyzed by XRD and found to be Sitagliptin dihydrophosphate Form II.

### Example 45

STG base Form I (100 mg) was slurried in iso-propyl acetate (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain wet STG phosphate crystalline form II.

### Example 46

STG phosphate (50 mg, crystalline form characterized by a powder XRD pattern with peaks at 4.7, 13.5, 17.7, 18.3, and 23.7±0.2 degrees two theta) was slurried in propylene glycol methyl ether (0.25 ml) at 25° C. for 16 hours.

The product was isolated by vacuum filtration to obtain a wet STG phosphate crystalline form II.

### Example 47

STG phosphate (50 mg, crystalline form characterized by a powder XRD pattern with peaks at 4.7, 13.5, 17.7, 18.3, and 23.7±0.2 degrees two theta) was slurried in chloroform (0.25 ml) at 25° C., then cooled gradually to 25° C. and stirred at 25° C. for 16 hours.

The product was isolated by vacuum filtration to obtain wet STG phosphate crystalline form II.

### Example 48

STG base Form I (100 mg) was slurried in iso-propyl acetate (1000 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by vacuum filtration to obtain a mixture of wet STG phosphate crystalline form II and form characterized by a powder XRD pattern with peaks at 4.7, 13.5, 17.7, 18.3, and 23.7±0.2 degrees two theta.

The sample was dried at 50° C. for 16 hours under reduced pressure to obtain a mixture of STG phosphate crystalline form characterized by a powder XRD pattern with peaks at 4.7, 13.5, 17.7, 18.3, and 23.7±0.2 degrees two theta.

### Example 49

Sitagliptin dihydrophosphate form characterized by a powder XRD pattern with peaks at 4.7, 13.5, 17.7, 18.3, and 23.7±0.2 degrees two theta (0.03 g) was slurried in 1 ml acetonitrile at 50° C., under magnetic stirring for 3 hours and at 10° C. for 16 hours. The product was isolated by filtration. The wet material was analyzed by XRD and found to be Sitagliptin dihydrophosphate Form II.

### Example 50

Sitagliptin dihydrophosphate form characterized by a powder XRD pattern with peaks at 4.7, 13.5, 17.7, 18.3, and 23.7±0.2 degrees two theta (0.03 g) was slurried in 0.3 ml ethanol at 25° C., under magnetic stirring for 24 hours. The product was isolated by filtration. The wet material was analyzed by XRD and found to be Sitagliptin dihydrophosphate Form II.

### Example 51

Sitagliptin dihydrophosphate form characterized by a powder XRD pattern with peaks at 4.7, 13.5, 17.7, 18.3, and 23.7±0.2 degrees two theta (0.03 g) was slurried in 0.3 ml iso-propyl alcohol at 25° C., under magnetic stirring for 24 hours. The product was isolated by filtration. The wet material was analyzed by XRD and found to be Sitagliptin dihydrophosphate Form II.

### Example 52

Sitagliptin dihydrophosphate form characterized by a powder XRD pattern with peaks at 4.7, 13.5, 17.7, 18.3, and 23.7±0.2 degrees two theta (0.03 g) was slurried in 0.3 ml diethylcarbonate at 25° C., under magnetic stirring for 24 hours. The product was isolated by filtration. The wet material was analyzed by XRD and found to be Sitagliptin dihydrophosphate Form II.

### Example 53

Sitagliptin dihydrophosphate form characterized by a powder XRD pattern with peaks at 4.7, 13.5, 17.7, 18.3, and 23.7±0.2 degrees two theta (0.03 g) was granulated with 0.006 ml isopropyl alcohol at 25° C., in a rotavapor for 9-12 hours. The wet material was analyzed by XRD and found to be Sitagliptin dihydrophosphate Form II.

### Example 54

STG phosphate (50 mg, crystalline form characterized by a powder XRD pattern with peaks at 4.7, 13.5, 17.7, 18.3, and 23.7±0.2 degrees two theta) was dissolved in dimethylsulfoxide (0.05 ml) at 25° C. Then Methyl iso-Butyl Ketone (1 ml) was added at room temperature. The solution formed was slurry (crystallization occurred) and was cooled in ice water bath for 2 hours.

The product was isolated by vacuum filtration to obtain STG phosphate form characterized by a powder XRD pattern with peaks at 4.7, 13.5, 17.7, 18.3, and 23.7±0.2 degrees two theta.

### Example 55

STG phosphate (50 mg, crystalline form characterized by a powder XRD pattern with peaks at 4.7, 13.5, 17.7, 18.3, and 23.7±0.2 degrees two theta) was dissolved in dimethylsulfoxide (0.05 ml) at 25° C. Then Tetrahydrofuran (1 ml) was added at 25° C. The solution formed was slurry (crystallization occurred) and was cooled in ice water bath for 16 hours.

The product was isolated by vacuum filtration to obtain wet amorphous STG phosphate.

### Example 56

STG phosphate (50 mg, crystalline form characterized by a powder XRD pattern with peaks at 4.7, 13.5, 17.7, 18.3, and 23.7±0.2 degrees two theta) was dissolved in dimethylsulfoxide (0.05 ml) at 25° C. Then methyl t-butyl ether (1 ml) was added at 25° C. The solution formed was slurry (crystallization occurred) and was cooled in ice water bath for 16 hours.

The product was isolated by vacuum filtration to obtain wet amorphous STG phosphate.

The sample was dried at 50° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 57

STG phosphate (50 mg, crystalline form characterized by a powder XRD pattern with peaks at 4.7, 13.5, 17.7, 18.3, and 23.7±0.2 degrees two theta) was slurried in water (300 μL) at 25° C., then heated to 60° C. and was dissolved at that temperature.

Then methyl t-butyl ether (250 μL) was added and the solution was cooled in an ice water bath, and stirred for 2 hours. Crystallization occurred.

The product was isolated by vacuum filtration to obtain wet STG phosphate monohydrate.

### Example 58

STG base Form I (100 mg) was dissolved in acetonitrile:water 1:1 (300 μL) at 25° C. Phosphoric acid (85%, 17 μL, 1 eq) was then added and the mixture was heated to 70° C., stirred at 70° C. for 2 hours, then cooled gradually to 25° C. and stirred at 25° C. for 16 hours. The product was isolated by evaporation to obtain wet STG phosphate crystalline monohydrate.

### Example 59

Sitagliptin dihydrophosphate form V, characterized by a powder XRD pattern with peaks at 4.7, 13.5, 17.7, 18.3, and 23.7±0.2 degrees two theta (0.03 g) was granulated with 0.006 ml

Iso-propanol:water 1:1 at 25° C., in a rotavapor for 9-12 hours. The wet material was analyzed by XRD and found to be Sitagliptin dihydrophosphate monohydrate.

### Example 60

STG base Form I (500 mg) was slurried in acetonitrile (2.5 mL) at 25° C. Phosphoric acid (85%, 83 μL, 1 eq) was then added and the mixture was stirred at 25° C. for 35 minutes. The product was isolated by vacuum filtration to obtain wet STG phosphate crystalline form II.

### Example 61

STG base Form I (500 mg) was slurried in toluene (2.5 mL) at 25° C. Phosphoric acid (85%, 83 μL, 1 eq) was then added and the mixture was stirred at 25° C. for 12 minutes. The product was isolated by vacuum filtration to obtain wet STG phosphate crystalline form II.

The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 62

STG base Form I (500 mg) was slurried in acetonitrile (1 mL) at 70° C. Phosphoric acid (85%, 83 μL, 1 eq) was then added and the mixture was stirred at 70° C. for 10 minutes. The product was isolated by vacuum filtration to obtain wet STG phosphate crystalline form II.

The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 63

STG base Form I (500 mg) was slurried in diethyl carbonate (2.5 mL) at 25° C. Phosphoric acid (85%, 83 μL, 1 eq) was then added and the mixture was stirred at 25° C. for 10 minutes.

The product was isolated by vacuum filtration to obtain wet STG phosphate amorphous.

### Example 64

STG base Form I (500 mg) was slurried in isobutyl acetate (2.5 mL) at 25° C. Phosphoric acid (85%, 83 μL, 1 eq) was then added and the mixture was stirred at 25° C. for 10 minutes.

The product was isolated by vacuum filtration to obtain wet STG phosphate form II.

### Example 65

STG base Form I (500 mg) was slurried in n-butanol (2.5 mL) at 25° C. Phosphoric acid (85%, 83 μL, 1 eq) was then added and the mixture was stirred at 25° C. for 25 minutes. The product was isolated by vacuum filtration to obtain wet STG phosphate crystalline form II.

### Example 66

STG base Form I (500 mg) was slurried in 1-propanol (2.5 mL) at 25° C. Phosphoric acid (85%, 83 μL, 1 eq) was then added and the mixture was stirred at 25° C. for 18 minutes. The product was isolated by vacuum filtration to obtain wet STG phosphate form II.

### Example 67

STG base Form I (500 mg) was dissolved in dimethyl carbonate (2.5 mL) at 74° C. Phosphoric acid (85%, 83 μL, 1 eq) was then added and the mixture was stirred at 74° C. for 13 minutes.

The product was isolated by vacuum filtration to obtain wet STG phosphate form II.

### Example 68

STG base Form I (500 mg) was dissolved in diethyl carbonate (2.5 mL) at 74° C. Phosphoric acid (85%, 83 μL, 1 eq) was then added and the mixture was stirred at 74° C. for 20 minutes.

The product was isolated by vacuum filtration to obtain wet STG phosphate form II.

### Example 69

STG base Form I (500 mg) was slurried in isobutyl acetate (2.5 mL) at 74° C. Phosphoric acid (85%, 83 μL, 1 eq) was then added and the mixture was stirred at 74° C. for 30 minutes.

The product was isolated by vacuum filtration to obtain wet STG phosphate form characterized by a powder XRD pattern with peaks at 4.7, 13.5, 17.7, 18.3, and 23.7±0.2 degrees two theta.

### Example 70

STG base Form I (500 mg) was slurried in n-Butanol (2.5 mL) at 74° C. Phosphoric acid (85%, 83 μL, 1 eq) was then added and the mixture was stirred at 74° C. for 18 minutes. The product was isolated by vacuum filtration to obtain wet STG phosphate form II. The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 71

STG base Form I (500 mg) was slurried in 1-propanol (2.5 mL) at 74° C. Phosphoric acid (85%, 83 μL, 1 eq) was then added and the mixture was stirred at 74° C. for 23 minutes. The product was isolated by vacuum filtration to obtain wet STG phosphate crystalline form II.

The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 72

STG base Form I (500 mg) was slurried in methyl isobutyl ketone (2.5 mL) at 74° C. Phosphoric acid (85%, 83 μL, 1 eq) was then added and the mixture was stirred at 74° C. for 25 minutes.

The product was isolated by vacuum filtration to obtain wet STG phosphate form characterized by a powder XRD pattern with peaks at 4.7, 13.5, 17.7, 18.3, and 23.7±0.2 degrees two theta.

The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 73

STG base Form I (500 mg) was slurried in dimethyl carbonate (5.5 mL) at 50° C. Phosphoric acid (85%, 83 μL, 1 eq) was then added and the mixture was stirred at 50° C. for 8 minutes.

The product was isolated by vacuum filtration to obtain wet STG phosphate amorphous.

### Example 74

STG base Form I (500 mg) was slurried in diethyl carbonate (10 mL) at 50° C. Phosphoric acid (85%, 83 μL, 1 eq) was then added and the mixture was stirred at 50° C. for 15 minutes.

The product was isolated by vacuum filtration to obtain wet STG phosphate amorphous.

### Example 75

STG base Form I (500 mg) was slurried in n-butanol (3.5 mL) at 50° C. Phosphoric acid (85%, 83 μL, 1 eq) was then added and the mixture was stirred at 50° C. for 1.25 hours. The product was isolated by vacuum filtration to obtain wet STG phosphate form II.

### Example 76

STG base Form I (500 mg) was slurried in 1-propanol (3.5 mL) at 50° C. Phosphoric acid (85%, 83 μL, 1 eq) was then added and the mixture was stirred at 50° C. for 1.25 hours. The product was isolated by vacuum filtration to obtain wet STG phosphate form II.

### Example 77

STG base Form I (500 mg) was slurried in acetonitrile (1.5 mL) at 50° C. Phosphoric acid (85%, 83 μL, 1 eq) was then added and the mixture was stirred at 50° C. for 10 minutes. The product was isolated by vacuum filtration to obtain wet STG phosphate form II.

### Example 78

STG base Form I (500 mg) slurried in acetonitrile (1.5 mL) at 70° C. was added dropwise to phosphoric acid (85%, 83 μL, 1 eq) in acetonitrile (1.5 mL) at 70° C. The mixture was stirred at 70° C. for 10 minutes.

The product was isolated by vacuum filtration to obtain wet STG phosphate form II.

### Example 79

STG base Form I (500 mg) slurried in acetonitrile (1 mL) at 70° C. was added dropwise to phosphoric acid (85%, 83 μL, 1 eq) in toluene (2.5 mL) at 70° C. The mixture was stirred at 70° C. for 15 minutes.

The product was isolated by vacuum filtration to obtain wet STG phosphate form characterized by a powder XRD pattern with peaks at 4.7, 13.5, 17.7, 18.3, and 23.7±0.2 degrees two theta.

The sample was dried at 40° C. for 16 hours under reduced pressure to obtain STG phosphate form characterized by a powder XRD pattern with peaks at 4.7, 13.5, 17.7, 18.3, and 23.7±0.2 degrees two theta.

### Example 80

STG base Form I (500 mg) slurried in 1-propanol (1.5 mL) at 72° C. was added dropwise to phosphoric acid (85%, 83 μL, 1 eq) in 1-propanol (1.5 mL) at 70° C. The mixture was stirred at 70° C. for 15 minutes.

The product was isolated by vacuum filtration to obtain wet STG phosphate form II.

### Example 81

STG base Form I (500 mg) slurried in acetonitrile (2.5 mL) at 25° C. was added dropwise to phosphoric acid (85%, 83 μL, 1 eq) in n-Butanol (5 mL) at 25° C. The mixture was stirred at 25° C. for 15 minutes.

The product was isolated by vacuum filtration to obtain wet STG phosphate form II. The sample was dried in vacuum oven at 40° C. 16 hours to obtain STG phosphate crystalline form characterized by a powder XRD pattern with peaks at 4.7, 13.5, 17.7, 18.3, and 23.7±0.2 degrees two theta.

### Example 82

STG base Form I (500 mg) slurried in acetonitrile (2.5 mL) at 50° C. was added dropwise to phosphoric acid (85%, 83 μL, 1 eq) in n-Butanol (5 mL) at 50° C. The mixture was stirred at 50° C. for 35 minutes.

The product was isolated by vacuum filtration to obtain a mixture of wet STG phosphate crystalline form characterized by a powder XRD pattern with peaks at 4.7, 13.5, 17.7, 18.3, and 23.7±0.2 degrees two theta and form II. The sample was dried in vacuum oven at 40° C. 16 hours to obtain STG phosphate crystalline form characterized by a powder XRD pattern with peaks at 4.7, 13.5, 17.7, 18.3, and 23.7±0.2 degrees two theta.

### Example 83

STG base Form I (500 mg) slurried in dimethyl carbonate (2.5 mL) at 50° C. was added dropwise to phosphoric acid (85%, 83 μL, 1 eq) in n-Hexane (2.5 mL) at 50° C. The mixture was stirred at 50° C. for 10 minutes.

The product was isolated by vacuum filtration to obtain a mixture of wet STG phosphate crystalline form characterized by a powder XRD pattern with peaks at 4.7, 13.5, 17.7, 18.3, and 23.7±0.2 degrees two theta and form II. The sample was dried in vacuum oven at 40° C. 16 hours to obtain STG phosphate crystalline form characterized by a powder XRD pattern with peaks at 4.7, 13.5, 17.7, 18.3, and 23.7±0.2 degrees two theta.

### Example 84

STG base Form I (500 mg) slurried in cyclohexanone (5 mL) at 25° C. was added dropwise to phosphoric acid (85%, 83 μL, 1 eq) in methyl tert-butyl ether (1 mL) at 25° C. The mixture crystallized after 30 minutes and was stirred for 45 minutes at 25° C.

The product was isolated by vacuum filtration to obtain wet amorphous STG phosphate. The sample was dried in vacuum oven at 40° C. 16 hours to obtain STG phosphate crystalline form monohydrate.

### Example 85

STG base Form I (500 mg) was added in portions to phosphoric acid (85%, 83 μL, 1 eq) in cyclopentyl methyl ether (5 mL) at 25° C. The mixture was stirred at 25° C. for 25 minutes. The product was isolated by vacuum filtration to obtain a mixture of STG phosphate crystalline form characterized by a powder XRD pattern with peaks at 4.7, 13.5, 17.7, 18.3, and 23.7±0.2 degrees two theta and form II. The sample was dried in vacuum oven at 40° C. 16 hours to obtain STG phosphate crystalline form characterized by a powder XRD pattern with peaks at 4.7, 13.5, 17.7, 18.3, and 23.7±0.2 degrees two theta.

### Example 86

STG base Form I (500 mg) slurried in cyclohexanone (5 mL) at 25° C. was added dropwise to phosphoric acid (85%, 83 μL, 1 eq) in methyl tert-butyl ether (1 mL) at 25° C. The mixture crystallized after 30 minutes and was stirred for 3 hours and 20 minutes at 25° C. The product was isolated by vacuum filtration to obtain wet amorphous STG phosphate. The sample was dried in vacuum oven at 40° C. 16 hours to obtain STG phosphate crystalline form monohydrate.

### Example 87

STG base Form I (500 mg) slurried in cyclohexanone (5 mL) at 25° C. was added dropwise to phosphoric acid (85%, 83 μL, 1 eq) in methyl tert-butyl ether (1 mL) at 25° C. The mixture crystallized after 30 minutes and was stirred for 1 week at 25° C.

The product was isolated by vacuum filtration to obtain wet amorphous STG phosphate. The sample was dried in vacuum oven at 40° C. 16 hours to obtain STG phosphate crystalline form monohydrate.

### Example 88

STG base Form I (5.6 g, 13.8 mmol) was dissolved in ethanol-water (18 ml-13 ml) at 50° C. To that solution, 85%-H₃PO₄ (0.92 ml, 13.8 mmol) was added at once with stirring. The solution was at 64-68° C. for an hour, and then the stirred solution was cooled to 25° C. for 40 min. The product was precipitated after additional stirring at 25° C. for 20 minutes. Ethanol (90 ml) was added to suspension, and the suspension was stirred at 25° C. for 18 hours. The solid was filtered, washed with ethanol (12 ml), dried at 50° C. under vacuum for 7 hours to give STG phosphate (6.0 g). The solid was analyzed by XRD and found to be STG phosphate Form characterized by a powder XRD pattern with peaks at 4.7, 13.5, 17.7, 18.3, and 23.7±0.2 degrees two theta. The STG phosphate Form V, characterized by a powder XRD pattern with peaks at 4.7, 13.5, 17.7, 18.3, and 23.7±0.2 degrees two theta (30 mg) was placed in a 50 ml- beaker. The opened beaker was kept in closed 100 ml-vessel containing 20 ml of methyl tert-butyl ether at 25° C. for 40 days. The solid was analyzed by XRD and found to be STG phosphate Form characterized by a powder XRD pattern with peaks at 4.7, 13.5, 17.7, 18.3, and 23.7±0.2 degrees two theta with higher crystallinity.

### Example 89

STG base (500 mg) was slurried in butyl acetate (2.5 mL) at 25° C., and was added drop-wise to phosphoric acid (85%, 83 μL, 1 eq) in butyl acetate (3.5 mL) at 25° C. The mixture was stirred at 25° C. for 20 minutes. The product was isolated by vacuum filtration to obtain wet STG phosphate crystalline form characterized by a powder XRD pattern with peaks at 4.7, 13.5, 17.7, 18.3, and 23.7±0.2 degrees two theta. The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 90

STG base (800 mg) was dissolved in methanol (2 mL) at 25° C., and heated to 50° C. Phosphoric acid (85%, 131 μL, 1 eq) in methanol (1 mL) was then added drop-wise, and the mixture was stirred at 50° C. The solution formed a very thick slurry. Therefore, 9 ml methanol was added in portions, and then stirred at 50° C. for 1 hour and at 25° C. for 16 hours. The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 91

STG base (600 mg) was slurried in isopropanol (3 mL) at 25° C., and heated to 50° C. Phosphoric acid (85%, 100 μL, 1 eq) in isopropanol (1 mL) was then added drop-wise, and the mixture was stirred at 50° C. for 16 hours. The sample was dried at 40° C. for 16 hours under reduced pressure to obtain STG phosphate crystalline form characterized by a powder XRD pattern with peaks at 4.7, 13.5, 17.7, 18.3, and 23.7±0.2 degrees two theta.

### Example 92:

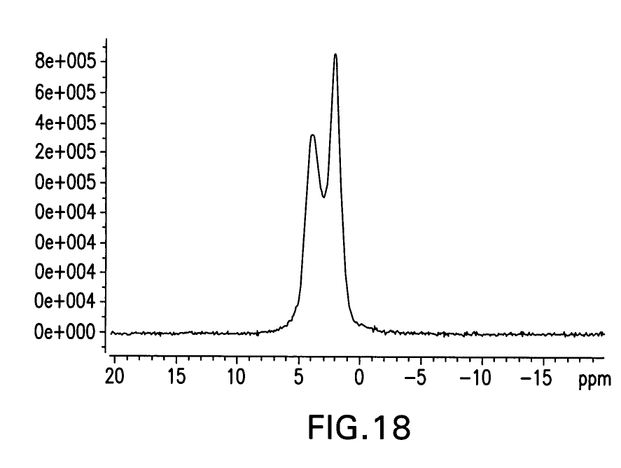

A 100 mg of a mixture of Form II and crystalline form characterized by a powder XRD pattern with peaks at 4.7, 13.5, 17.7, 18.3, and 23.7±0.2 degrees two theta was kept under relative humidity of 100% for one day, to obtain pure crystalline form characterized by a powder XRD pattern with peaks at 4.7, 13.5, 17.7, 18.3, and 23.7±0.2 degrees two theta, as presented in FIG. 18.

### Example 93

Sitagliptin phosphate (9 gr, a dry mixture of crystalline Form II and a form characterized by a powder XRD pattern with peaks at 4.7, 13.5, 17.7, 18.3, and 23.7±0.2 degrees two theta) was dried in fluidized bed dryer at 40° C. at 40% humidity for four hours to obtain Sitagliptin phosphate crystalline form characterized by a powder XRD pattern with peaks at 4.7, 13.5, 17.7, 18.3, and 23.7±0.2 degrees two theta (6.8 gr).

### Example 94

Sitagliptin phosphate (1 gr, a dry mixture of crystalline Form II and a form characterized by a powder XRD pattern with peaks at 4.7, 13.5, 17.7, 18.3, and 23.7±0.2 degrees two theta) was dried in vacuum oven at 80° C. for 24 hours to obtain Sitagliptin phosphate crystalline form characterized by a powder XRD pattern with peaks at 4.7, 13.5, 17.7, 18.3, and 23.7±0.2 degrees two theta.

### Example 95

Sitagliptin phosphate (1 gr, a dry mixture of crystalline Form II and a form characterized by a powder XRD pattern with peaks at 4.7, 13.5, 17.7, 18.3, and 23.7±0.2 degrees two theta) was dried in vacuum oven at 100° C. for 24 hours to obtain Sitagliptin phosphate crystalline form characterized by a powder XRD pattern with peaks at 4.7, 13.5, 17.7, 18.3, and 23.7±0.2 degrees two theta.

### Example 96

Sitagliptin phosphate from characterized by a powder XRD pattern with peaks at 4.7, 13.5, 17.7, 18.3, and 23.7±0.2 degrees two theta was stored under ethanol vapors at 25° C. for 18 hours. It was then analyzed by PXRD, and identified as form II of Sitagliptin phosphate.

### Example 97

Sitagliptin phosphate from characterized by a powder XRD pattern with peaks at 4.7, 13.5, 17.7, 18.3, and 23.7±0.2 degrees two theta was stored under methanol vapors at 25° C. for 1 week. It was then analyzed by PXRD, and identified as form II of Sitagliptin phosphate.

### Example 98

Sitagliptin phosphate from characterized by a powder XRD pattern with peaks at 4.7, 13.5, 17.7, 18.3, and 23.7±0.2 degrees two theta was stored under iso-propanol vapors at 25° C. for 1 week. It was then analyzed by PXRD, and identified as form II of Sitagliptin phosphate.

### Example 99

To 1 g of amorphous Sitagliptin-phosphate (97.8% purity and 81.9% R) was added 50 ml of acetonitrile (ACN). The slurry was heated to reflux and stirred for 1 hour, then cooled to 2° C., and stirred for 1 hour. The product was isolated by vacuum filtration at 2° C., and washed with 2 ml of ACN, and dried at 50° C. in a vacuum oven for 15 hours to yield 0.88 g of Sitagliptin-phosphate (100% purity and 75.5% R) form VI (88% yield).

### Example 100

To 5 g of oily STG-base (75.1 % R) was added 20 ml of isopropanol (IPA). The slurry was heated to 50° C., than H₃PO₄ 85% (1.13 g in 10 ml IPA) was added dropwise and stirred for 1 hour. The slurry reaction was cooled to room temperature, and stirred for three days. The product was isolated by vacuum filtration, and washed with 20 ml of IPA to yield STG-Phosphate form VI, as a white-grey solid (99.5% purity and 74.7% R). Further purification accepted by adding 50 ml of ACN to the product. The slurry mixture was heated to reflux and stirred for 1 to 2 hours, than cooled to room temperature and stirred over night. Vacuum filtration followed by washings with 40 ml ACN yield a white-grey solid that was dried at 40° C. in a vacuum oven for 15 hours to yield 4.74 g of STG-Phosphate (99.7% purity and 78.0% R) form VI (95% yield).

### Example 101

To degaussed 2,2,2-trifluoroethanol (TFE) (30 mL) were added Rhodium(I) chloride 1,5-cycloocatadiene complex (18.3 mg, 0.05%) and (R)-(−)-1-[(S)-2-diphenylphosphino)ferrocenyl]ethyl di-tert-butylphosphine (44.2 mg, 0.11%). The solution was stirred at room temperature, degaussed three times, and then stirred for one hour at room temperature.

To 250 ml hydrogenator were added (Z)-3-amino-1-(3-(trifluoromethyl)-5,6-dihydro-[1,2,4]triazolo[4,3-a]pyrazyn-7(8H)-yl)-4-(2,4,5-trifluorophenyl)but-2-en-1-one (30 gr, 1 equivalent) and TFE (120 ml) at room temperature and the mixture was washed three times with nitrogen gas. The catalyst solution was added and the clear solution was washed three times with nitrogen gas and then with hydrogen gas. The mixture remained under hydrogen at constant pressure of 5 bar and heated to 55° C. The mixture was stirred at 55° C. for 26 hours to obtain Sitagliptin base in TFE solution (optical purity by HPLC 76.9%, purity by HPLC 91.5%)

Two reaction mixtures which were obtained according to the above procedure were combined and the solution was divided to 10 parts.

7 parts of the solution, each contained ca ˜6 gr Sitagliptin were concentrated and Sitagliptin base was precipitated by addition of MTBE then filtrated by vacuum filtration. The combined mother liqueur from the crystallization experiments was concentrated. The residue was dissolved in isopropanol (40 mL) at room temperature, heated to 50° C. A solution of phosphoric acid (85%, 1.7 mL, ca ˜1 eq) in isopropanol (20 mL) was added and the mixture kept stirring at 50° C. for one hour, then cooled gradually to 25° C., and stirred at 25° C. over night.

The product was isolated by vacuum filtration and dried at 40° C. vacuum oven over night to obtain Sitagliptin phosphate crystalline form VI (optical purity by HPLC 51.8%, purity by HPLC 99.20%).

What is claimed:

1. A process for preparing a crystalline form of Sitagliptin phosphate, characterized by a powder XRD pattern with peaks at about 4.7, 13.5, 17.7, 18.3, and 23.7±0.2 degrees two theta, the process selected from the group consisting of:
a. a process comprising combining Sitagliptin base, phosphoric acid, and a solvent selected from the group consisting of ethyl acetate, dioxane, methyl isobutyl ketone, isobutyl acetate, butyl acetate, a mixture of acetonitrile and toluene, or a mixture of tetrahydrofuran and water to form a slurry; and obtaining a Sitagliptin phosphate precipitate;
b. a process comprising combining Sitagliptin phosphate or Sitagliptin base and phosphoric acid with a mixture of a first organic solvent and a second organic solvent selected from the group consisting of acetone:n-hexane, acetone:n-heptane, acetone:cyclopentyl methyl ether, acetone:dibutyl ether, acetone:isopropylacetate, dimethylsulfoxide:methyl isobutyl ketone, and dimethylsulfoxide:methyl tert butyl ether, forming a mixture, and crystallizing Sitagliptin phosphate from the mixture, wherein, when acetone:cyclopentyl methyl ether, acetone:isopropylacetate, and dimethylsulfoxide:methyl tert butyl ether, are used, the crystallized Sitagliptin phosphate is further dried;
c. a process comprising drying wet Sitagliptin phosphate Form II;
d. a process comprising heating a mixture of Sitagliptin phosphate Form II and the crystalline form characterized by a powder XRD pattern with peaks at about 4.7, 13.5, 17.7, 18.3, and 23.7±0.2 degrees two theta, to a temperature of about 40° C. to about 100° C. under reduced pressure; and
e. a process comprising drying a mixture of Sitagliptin phosphate Form II and a crystalline form, characterized by a powder XRD pattern with peaks at about 4.7, 13.5, 17.7, 18.3, and 23.7±0.2 degrees two theta, in a fluidized bed dryer at a temperature of about 30° C. to about 60° C.

2. The process of claim 1, wherein Sitagliptin phosphate or Sitagliptin base and phosphoric acid are combined with the mixture of the first organic solvent and the second organic solvent.

3. The process of claim 2, wherein the first organic solvent and the second organic solvent ratio is about 1:1 to about 1:15.

4. The process of claim 2, wherein Sitagliptin phosphate is combined with the mixture of the first organic solvent and the second organic solvent.

5. The process of claim 2, wherein the solution is heated to a temperature of about 45° C. to about 80° C.

6. The process of claim 1, wherein the wet Sitagliptin phosphate Form II is dried.

7. The process of claim 6, wherein the wet Sitagliptin phosphate Form II is dried at about 40° C. to about 100° C. under reduced pressure.

8. The process of claim 6, wherein the Sitagliptin phosphate Form II is prepared in a process, comprising combining Sitagliptin base and phosphoric acid and an organic solvent selected from the group consisting of dimethyl carbonate, tetrahydrofuran, propylene glycol methyl ether, methyl ethyl ketone, ethanol, methyl acetate, dimethylformamide, diethyl carbonate, n-butanol, 1-propanol, toluene, isobutyl acetate, isopropyl acetate, isopropanol, and a mixture of acetonitrile and n-butanol, acetonitrile, dimethyl carbonate to form a slurry, and obtaining Sitagliptin phosphate Form II.

9. The process of claim 6, wherein the Sitagliptin phosphate Form II is prepared in a process comprising combining Sitagliptin base and phosphoric acid and a mixture of a first organic solvent and a second organic solvent selected from the group consisting of acetone:isopropylacetate, acetone:cyclohexane, acetone:isobutyl acetate, acetonitrile:n-butanol, and acetone:n-butanol, forming a mixture, and obtaining Sitagliptin phosphate Form II.

10. The process of claim 9, wherein the first organic solvent and the second organic solvent ratio is about 1:1 to about 1:15.

11. The process of claim 6, wherein the wet Sitagliptin phosphate Form II comprises a solvent selected from the group consisting of methyl isobutyl ketone, dimethyl carbonate, tetrahydrofuran, acetonitrile, propylene glycol methyl ether, methanol, n-butanol, 1-propanol, toluene, isobutyl acetate, isopropyl acetate, butyl acetate, isopropanol, dimethyl carbonate, n-hexane, acetone, cyclohexane, isobutyl acetate, and mixtures thereof.

12. The process of claim 1, wherein the mixture of Sitagliptin phosphate Form II and the crystalline form characterized by a powder XRD pattern with peaks at about 4.7, 13.5, 17.7, 18.3, and 23.7±0.2 degrees two theta are heated to a temperature of about 40° C. to about 100° C. under reduced pressure.

13. The process of claim 12, wherein the mixture is heated at a temperature of about 40° C. to about 60° C.

14. The process of claim 12, wherein said mixture is heated for about 10 to about 24 hours.

15. The process of claim 1, wherein the mixture of Sitagliptin phosphate Form II and the crystalline form, characterized by a powder XRD pattern with peaks at about 4.7, 13.5, 17.7, 18.3, and 23.7±0.2 degrees two theta, is dried in a fluidized bed dryer at a temperature of about 30° C. to about 60° C.

16. A process for preparing Sitagliptin phosphate Form II, the process selected from the group consisting of:
a. a process comprising providing a slurry of crystalline Sitagliptin phosphate, characterized by a powder XRD pattern with peaks at about 4.7, 13.5, 17.7, 18.3, and 23.7±0.2 degrees two theta, and a solvent selected from the group consisting of acetonitrile, methanol, ethanol, 1-propanol, isopropanol, acetone, tetrahydrofuran, n-butanol, iso-butanol, toluene, propylene glycol, propylene glycol methyl ether, chloroform, diethyl carbonate, dimethylformamide, or mixtures of dimethylformamide with methyl isobutyl ketone, or n-butanol; heating the slurry; and obtaining Sitagliptin phosphate Form II;
b. a process comprising combining Sitagliptin base and phosphoric acid in an organic solvent selected from the group consisting of dimethyl carbonate, tetrahydrofuran, propylene glycol methyl ether, methyl ethyl ketone, ethanol, methyl acetate, dimethylformamide, diethyl carbonate, n-butanol, 1-propanol, toluene, isobutyl acetate, isopropyl acetate, isopropanol, a mixture of acetonitrile and n-butanol, acetonitrile, dimethyl carbonate, and a mixture of dimethyl carbonate and n-hexane, forming a slurry; and obtaining Sitagliptin phosphate Form II;
c. a process comprising combining Sitagliptin base and phosphoric acid and a mixture of a first organic solvent and a second organic solvent selected from the group consisting of acetone:isopropylacetate, acetone:cyclohexane, acetone:isobutyl acetate, acetonitrile:n-butanol, and acetone:n-butanol, forming a mixture; and crystallizing Sitagliptin phosphate from the mixture, obtaining Sitagliptin phosphate Form II;
d. a process comprising dissolving Sitagliptin phosphate, characterized by a powder XRD pattern with peaks at about 4.7, 13.5, 17.7, 18.3, and 23.7±0.2 degrees two theta, in dimethylsulfoxide; and adding an antisolvent selected from the group consisting of iso-butanol, acetonitrile, diethyl ether, diethyl carbonate, and tert-butyl ether;
e. a process comprising granulating a crystalline Sitagliptin phosphate, characterized by a powder XRD pattern with peaks at about 4.7, 13.5, 17.7, 18.3, and 23.7±0.2 degrees two theta in the presence of isopropanol; and
f. a process comprising exposing crystalline Sitagliptin phosphate, characterized by a powder XRD pattern with peaks at about 4.7, 13.5, 17.7, 18.3, and 23.7±0.2 degrees two theta to a C₁-C₄ alcohol, preferably the alcohol is selected from the group consisting of ethanol, methanol, and isopropanol.

17. The process of claim 16, wherein the slurry is heated to about 50° C. to about 80° C.

18. The process of claim 16, wherein the Sitagliptin base and phosphoric acid are combined in an organic solvent selected from the group consisting of dimethyl carbonate, tetrahydrofuran, propylene glycol methyl ether, methyl ethyl ketone, ethanol, methyl acetate, dimethylformamide, diethyl carbonate, n-butanol, 1-propanol, toluene, isobutyl acetate, isopropyl acetate, isopropanol, a mixture of acetonitrile and n-butanol, acetonitrile, dimethyl carbonate, and a mixture of dimethyl carbonate and n-hexane, forming a slurry.

19. The process of claim 16, wherein the Sitagliptin base and phosphoric acid and a mixture of a first organic solvent and a second organic solvent selected from the group consisting of acetone:isopropylacetate, acetone:cyclohexane, acetone:isobutyl acetate, acetonitrile:n-butanol, and acetone:n-butanol are combined forming a mixture; and Sitagliptin phosphate is crystallized from the mixture.

20. The process of claim 19, wherein the first organic solvent and the second organic solvent ratio is about 1:1 to about 1:15.

21. The process of claim 16, wherein the Sitagliptin phosphate, characterized by a powder XRD pattern with peaks at about 4.7, 13.5, 17.7, 18.3, and 23.7±0.2 degrees two theta, is dissolved in dimethylsulfoxide and an antisolvent selected from the group consisting of iso-butanol, acetonitrile, diethyl ether, diethyl carbonate, and tert-butyl ether is added.

22. The process of claim 16, wherein crystalline Sitagliptin phosphate, characterized by a powder XRD pattern with peaks at about 4.7, 13.5, 17.7, 18.3, and 23.7±0.2 degrees two theta, is granulated in the presence of isopropanol.

23. The process of claim 22, wherein the solvent/antisolvent ration is about 1:1 to about 1:20.

24. The process of claim 22, wherein the solvent/antisolvent ration is about 3:10.

25. The process of claim 16, wherein crystalline Sitagliptin phosphate, characterized by a powder XRD pattern with peaks at about 4.7, 13.5, 17.7, 18.3, and 23.7±0.2 degrees two theta, is exposed to a C₁-C₄ alcohol.

26. The process of claim 25, wherein the alcohol is selected from the group consisting of ethanol, methanol, and isopropanol.
